(19) United States
(12) Patent Application Publication
(10) Pub. No.: US 2022/0213078 A1
(43) Pub. Date: Jul. 7, 2022

(54) MODULATORS OF THE INTEGRATED STRESS RESPONSE PATHWAY

(71) Applicant: EVOTEC INTERNATIONAL GMBH, Hamburg (DE)

(72) Inventors: Christopher John BROWN, Abingdon (GB); Irena Doly REBOULE, Abingdon (GB); York RUDHARD, Hamburg (DE); Mohamad SABBAH, Abingdon (GB); Daryl Simon WALTER, Abingdon (GB)

(21) Appl. No.: 17/605,369

(22) PCT Filed: Apr. 22, 2020
(86) PCT No.: PCT/EP2020/061148

§ 371 Date: Oct. 21, 2021

(30) Foreign Application Priority Data
Apr. 24, 2019 (EP) 19170502.9

(51) Int. Cl. C07D 413/04; C07D 413/14; A61K 45/06; A61K 31/4245; A61K 31/4439; A61K 31/506; A61K 31/497

(52) U.S. Cl. CPC C07D 413/04; C07D 413/14; A61K 45/06; A61K 31/497; A61K 31/4439; A61K 31/506; A61K 31/4245

(57) ABSTRACT

The present invention relates to compounds of formula (I) or pharmaceutically acceptable salts, solvates, hydrates, tautomers or stereoisomers thereof, wherein R¹ to R³, A¹ and A² have the meaning as indicated in the description and claims. The invention further relates to pharmaceutical compositions comprising said compounds, their use as medicament and in a method for treating and preventing one or more diseases or disorders associated with integrated stress response.

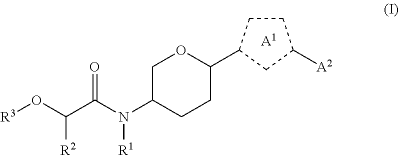

The present invention relates to compounds of formula (I)

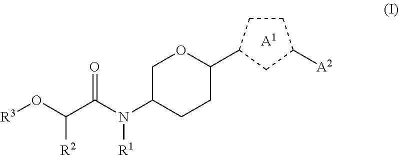

or pharmaceutically acceptable salts, solvates, hydrates, tautomers or stereoisomers thereof, wherein R¹ to R³, A¹ and A² have the meaning as indicated in the description and claims. The invention further relates to pharmaceutical compositions comprising said compounds, their use as medicament and in a method for treating and preventing one or more diseases or disorders associated with integrated stress response.

The Integrated Stress Response (ISR) is a cellular stress response common to all eukaryotes (1). Dysregulation of ISR signaling has important pathological consequences linked inter alia to inflammation, viral infection, diabetes, cancer and neurodegenerative diseases. ISR is a common denominator of different types of cellular stresses resulting in phosphorylation of the alpha subunit of eukaryotic translation initiation factor 2 (eIF2alpha) on serine 51 leading to the suppression of normal protein synthesis and expression of stress response genes (2). In mammalian cells the phosphorylation is carried out by a family of four eIF2alpha kinases, namely: PKR-like ER kinase (PERK), double-stranded RNA-dependent protein kinase (PKR), heme-regulated eIF2alpha kinase (HRI), and general control nonderepressible 2 (GCN2), each responding to distinct environmental and physiological stresses (3).

eIF2alpha together with eIF2beta and eIF2gamma form the eIF2 complex, a key player of the initiation of normal mRNA translation (4). The eIF2 complex binds GTP and Met-tRNAi forming a ternary complex (eIF2-GTP-Met-tRNAi), which is recruited by ribosomes for translation initiation (5, 6).

eIF2B is a heterodecameric complex consisting of 5 subunits (alpha, beta, gamma, delta, epsilon) which in duplicate form a GEF-active decamer (7).

In response to ISR activation, phosphorylated eIF2alpha inhibits the eIF2B-mediated exchange of GDP for GTP, resulting in reduced ternary complex formation and hence in the inhibition of translation of normal mRNAs characterized by ribosomes binding to the 5′ AUG start codon (8). Under these conditions of reduced ternary complex abundance the translation of several specific mRNAs including the mRNA coding for the transcription factor ATF4 is activated via a mechanism involving altered translation of upstream ORFs (uORFs) (7, 9, 10).

These mRNAs typically contain one or more uORFs that normally function in unstressed cells to limit the flow of ribosomes to the main coding ORF. For example, during normal conditions, uORFs in the 5′ UTR of ATF occupy the ribosomes and prevent translation of the coding sequence of ATF4. However, during stress conditions, i.e. under conditions of reduced ternary complex formation, the probability for ribosomes to scan past these upstream ORFs and initiate translation at the ATF4 coding ORF is increased. ATF4 and other stress response factors expressed in this way subsequently govern the expression of an array of further stress response genes. The acute phase consists in expression of proteins that aim to restore homeostasis, while the chronic phase leads to expression of pro-apoptotic factors (1, 11, 12, 13).

Upregulation of markers of ISR signaling has been demonstrated in a variety of conditions, among these cancer and neurodegenerative diseases. In cancer, ER stress-regulated translation increases tolerance to hypoxic conditions and promotes tumor growth (14, 15, 16), and deletion of PERK by gene targeting has been shown to slow growth of tumours derived from transformed PERK^{-/-} mouse embryonic fibroblasts (14, 17). Further, a recent report has provided proof of concept using patient derived xenograft modeling in mice for activators of eIF2B to be effective in treating a form of aggressive metastatic prostate cancer (28). Taken together, prevention of cytoprotective ISR signaling may represent an effective antiproliferation strategy for the treatment of at least some forms of cancer.

Further, modulation of ISR signaling could prove effective in preserving synaptic function and reducing neuronal decline, also in neurodegenerative diseases that are characterized by misfolded proteins and activation of the unfolded protein response (UPR), such as amyotrophic lateral sclerosis (ALS), frontotemporal dementia (FTD), Alzheimer's disease (AD), Parkinson's disease (PD) and Jakob Creutzfeld (prion) diseases (18, 19, 20). With prion disease an example of a neurodegenerative disease exists where it has been shown that pharmacological as well as genetic inhibition of ISR signaling can normalize protein translation levels, rescue synaptic function and prevent neuronal loss (21). Specifically, reduction of levels of phosphorylated eIF2alpha by overexpression of the phosphatase controlling phosphorylated eIF2alpha levels increased survival of prion-infected mice whereas sustained eIF2alpha phosphorylation decreased survival (22).

Further, direct evidence for the importance of control of protein expression levels for proper brain function exists in the form of rare genetic diseases affecting functions of eIF2 and eIF2B. A mutation in eIF2gamma that disrupts complex integrity of eIF2 and hence results in reduced normal protein expression levels is linked to intellectual disability syndrome (ID) (23). Partial loss of function mutations in subunits of eIF2B have been shown to be causal for the rare leukodystrophy Vanishing White Matter Disease (VWMD) (24, 25). Specifically, stabilization of eIF2B partial loss of function in a VWMD mouse model by a small molecule related to ISRIB has been shown to reduce ISR markers and improve functional as well as pathological end points (26, 27).

Modulators of the eIF2 alpha pathway are described in WO 2014/144952 A2. WO 2017/193030 A1, WO 2017/193034 A1, WO 2017/193041 A1 and WO 2017/193063 A1 describe modulators of the integrated stress pathway. WO 2017/212423 A1, WO 2017/212425 A1, WO 2018/225093 A1, WO 2019/008506 A1 and WO 2019/008507 A1 describe inhibitors of the ATF4 pathway. WO 2019/032743 A1 and WO 2019/046779 A1 relate to eukaryotic initiation factor 2B modulators.

Further documents describing modulators of the integrated stress pathway are WO 2019/090069 A1, WO 2019/090074 A1, WO 2019/090076 A1, WO 2019/090078 A1, WO 2019/090081 A1, WO 2019/090082 A1, WO 2019/090085 A1, WO 2019/090088 A1, WO 2019/090090 A1. Modulators of eukaryotic initiation factors are described in WO 2019/183589 A1. WO 2019/118785 A2 describes inhibitors of the integrated stress response pathway. Heteroaryl derivatives as ATF4 inhibitors are described in WO 2019/193540 A1. Bicyclic aromatic ring derivatives as ATF4 inhibitors are described in WO 2019/193541 A1.

However, there is a continuing need for new compounds useful as modulators of the integrated stress response pathway with good pharmacokinetic properties.

Thus, an object of the present invention is to provide a new class of compounds as modulators of the integrated stress response pathway, which may be effective in the treatment of integrated stress response pathway related diseases and which may show improved pharmaceutically relevant properties including activity, selectivity, ADMET properties and/or reduced side effects.

Accordingly, the present invention provides a compound of formula (I)

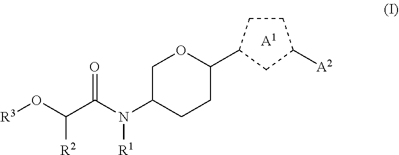

or a pharmaceutically acceptable salt, solvate, hydrate, tautomer or stereoisomer thereof, wherein

A¹ is C₅ cycloalkylene, C₅ cycloalkenylene, or a nitrogen ring atom containing 5-membered heterocyclene, wherein A¹ is optionally substituted with one or more R⁴, which are the same or different;

each R⁴ is independently halogen, CN, OR⁵ oxo (═O) where the ring is at least partially saturated or C₁₋₆ alkyl, wherein C₁₋₆ alkyl is optionally substituted with one or more halogen, which are the same or different;

R⁵ is H or C₁₋₆ alkyl, wherein C₁₋₆ alkyl is optionally substituted with one or more halogen, which are the same or different;

A² is phenyl or 5- to 6-membered aromatic heterocyclyl, preferably phenyl or 6-membered aromatic heterocyclyl, wherein A² is optionally substituted with one or more R⁶, which are the same or different;

each R⁶ is independently OH, O(C₁₋₆ alkyl), halogen, CN, cyclopropyl, C₁₋₆ alkyl, C₂₋₆ alkenyl, or C₂₋₆ alkynyl, wherein cyclopropyl, C₁₋₆ alkyl, C₂₋₆ alkenyl, and C₂₋₆ alkynyl are optionally substituted with one or more halogen, which are the same or different; or two R⁶ are joined to form together with atoms to which they are attached a ring A^{2a};

A^{2a} is phenyl; C₃₋₇ cycloalkyl; or 3 to 7 membered heterocyclyl, wherein A^{2a} is optionally substituted with one or more R⁷, which are the same or different;

each R⁷ is independently C₁₋₆ alkyl, C₂₋₆ alkenyl or C₂₋₆ alkynyl, wherein C₁₋₆ alkyl, C₂₋₆ alkenyl, and C₂₋₆ alkynyl are optionally substituted with one or more halogen, which are the same or different;

R¹ is H or C₁₋₄ alkyl, preferably H, wherein C₁₋₄ alkyl is optionally substituted with one or more halogen, which are the same or different;

R² is H or C₁₋₄ alkyl, wherein C₁₋₄ alkyl is optionally substituted with one or more halogen, which are the same or different; and

R³ is A³; or

R² and R³ are joined to form a 3,4-dihydro-2H-1-benzopyran ring, which is optionally substituted with one or more R⁸, which are the same or different;

A³ is phenyl or 5- to 6-membered aromatic heterocyclyl, preferably, phenyl or 6-membered aromatic heterocyclyl, wherein A³ is optionally substituted with one or more R⁸, which are the same or different;

each R⁸ is independently halogen, CN, C(O)OR⁹, OR⁹, C(O)R⁹, C(O)N(R⁹R^{9a}) S(O)₂N(R⁹R^{9a}), S(O)N(R⁹R^{9a}), S(O)₂R⁹, S(O)R⁹, N(R⁹)S(O)₂N(R^{9a}R^{9b}), SR⁹, N(R⁹R^{9a}), NO₂, OC(O)R⁹, N(R⁹)C(O)R^{9a}, N(R⁹)S(O)₂R^{9a}, N(R⁹)S(O)R^{9a}, N(R⁹)C(O)OR^{9a} N(R⁹)C(O)N(R^{9a}R^{9b}), OC(O)N(R⁹R^{9a}), oxo (═O) where the ring is at least partially saturated, C₁₋₆ alkyl, C₂₋₆ alkenyl, or C₂₋₆ alkynyl, wherein C₁₋₆ alkyl, C₂₋₆ alkenyl, and C₂₋₆ alkynyl are optionally substituted with one or more R¹⁰, which are the same or different; R⁹, R^{9a}, R^{9b} are independently selected from the group consisting of H, C₁₋₆ alkyl, C₂₋₆ alkenyl, and C₂₋₆ alkynyl, wherein C₁₋₆ alkyl, C₂₋₆ alkenyl, and C₂₋₆ alkynyl are optionally substituted with one or more halogen, which are the same or different;

each R¹⁰ is independently halogen, CN, C(O)OR¹¹, OR¹¹, C(O)R¹¹, C(O)N(R¹¹R^{11a}) S(O)₂N(R¹¹R^{11a}), S(O)N(R¹¹R^{11a}), S(O)₂R¹¹, S(O)R¹¹, N(R¹¹)S(O)₂N(R^{11a}R^{11b}), SR¹¹, N(R¹¹R^{11a}), NO₂, OC(O)R¹¹, N(R¹¹)C(O)R^{11a}, N(R¹¹)SO₂R^{11a} N(R¹¹)S(O)R^{11a}, N(R¹¹)C(O)N(R^{11a}R^{11b}), N(R¹¹)C(O)OR^{11a}, or OC(O)N(R¹¹R^{11a});

R¹¹, R^{11a}, R^{11b} are independently selected from the group consisting of H, C₁₋₆ alkyl, C₂₋₆ alkenyl, and C₂₋₆ alkynyl, wherein C₁₋₆ alkyl, C₂₋₆ alkenyl, and C₂₋₆ alkynyl are optionally substituted with one or more halogen, which are the same or different.

In case a variable or substituent can be selected from a group of different variants and such variable or substituent occurs more than once the respective variants can be the same or different.

Within the meaning of the present invention the terms are used as follows:

The term “optionally substituted” means unsubstituted or substituted. Generally—but not limited to—, “one or more substituents” means one, two or three, preferably one or two substituents and more preferably one substituent. Generally these substituents can be the same or different.

“Alkyl” means a straight-chain or branched hydrocarbon chain. Each hydrogen of an alkyl carbon may be replaced by a substituent as further specified.

“Alkenyl” means a straight-chain or branched hydrocarbon chain that contains at least one carbon-carbon double bond. Each hydrogen of an alkenyl carbon may be replaced by a substituent as further specified.

“Alkynyl” means a straight-chain or branched hydrocarbon chain that contains at least one carbon-carbon triple bond. Each hydrogen of an alkynyl carbon may be replaced by a substituent as further specified.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl, tert-butyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent as further specified.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl, tert-butyl, n-pentyl, n-hexyl, or e.g. —CH₂—, —CH₂—CH₂—, —CH(CH₃)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent as further specified.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₆ alkenyl carbon may be replaced by a substituent as further specified.

“C₂₋₆ alkynyl” means an alkynyl chain having 2 to 6 carbon atoms, e.g. if present at the end of a molecule: —C═CH, —CH₂—C═CH, CH₂—CH₂—C═CH, CH₂—C═C—CH₃, or e.g. —C═C— when two moieties of a molecule are linked by the alkynyl group. Each hydrogen of a C₂₋₆ alkynyl carbon may be replaced by a substituent as further specified.

“C₃₋₇ cycloalkyl” or “C₃₋₇ cycloalkyl ring” means a cyclic alkyl chain having 3-7 carbon atoms, e.g. cyclopropyl, cyclobutyl, cyclopentyl, cyclohexyl, cyclohexenyl, cycloheptyl. Preferably, cycloalkyl refers to cyclopropyl, cyclobutyl, cyclopentyl, cyclohexyl, or cycloheptyl. Each hydrogen of a cycloalkyl carbon may be replaced by a substituent as further specified herein. The term “C₃₋₅ cycloalkyl” or “C₃₋₅ cycloalkyl ring” is defined accordingly.

“C₅ cycloalkylene” refers to a bivalent cycloalkyl with five carbon atoms, i.e. a bivalent cyclopentyl ring.

“C₅ cycloalkenylene” refers to a bivalent cycloalkenylene, i.e. a bivalent cyclopentene or cyclopentadiene.

“Halogen” means fluoro, chloro, bromo or iodo. It is generally preferred that halogen is fluoro or chloro.

“3 to 7 membered heterocyclyl” or “3 to 7 membered heterocycle” means a ring with 3, 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 3 to 7 membered heterocycle are aziridine, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine. The term “5 to 6 membered heterocyclyl” or “5 to 6 membered heterocycle” is defined accordingly. The term “5 membered heterocyclyl” or “5 membered heterocycle” is defined accordingly and includes 5 membered aromatic heterocyclyl or heterocycle.

The term “nitrogen ring atom containing 5-membered heterocyclene” refers to a bivalent 5-membered heterocycle, wherein at least one of the five ring atoms is a nitrogen atom and wherein the ring is linked to the rest of the molecule via a carbon or nitrogen atom.

“Saturated 4 to 7 membered heterocyclyl” or “saturated 4 to 7 membered heterocycle” means fully saturated “4 to 7 membered heterocyclyl” or “4 to 7 membered heterocycle”.

“4 to 7 membered at least partly saturated heterocyclyl” or “4 to 7 membered at least partly saturated heterocycle” means an at least partly saturated “4 to 7 membered heterocyclyl” or “4 to 7 membered heterocycle”.

“5 to 6 membered aromatic heterocyclyl” or “5 to 6 membered aromatic heterocycle” means a heterocycle derived from cyclopentadienyl or benzene, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole, pyridine, pyrimidine, pyridazine, pyrazine, triazine.

“5 membered aromatic heterocyclyl” or “5 membered aromatic heterocycle” means a heterocycle derived from cyclopentadienyl, where at least one carbon atom is replaced by a heteroatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, triazole, tetrazole.

“7 to 12 membered heterobicyclyl” or “7 to 12 membered heterobicycle” means a heterocyclic system of two rings with 7 to 12 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 7 to 12 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine or pteridine. The term 7 to 12 membered heterobicycle also includes spiro structures of two rings like 6-oxa-2-azaspiro[3,4]octane, 2-oxa-6-azaspiro[3.3]heptan-6-yl or 2,6-diazaspiro[3.3]heptan-6-yl or bridged heterocycles like 8-aza-bicyclo[3.2.1]octane or 2,5-diazabicyclo[2.2.2]octan-2-yl or 3,8-diazabicyclo[3.2.1]octane.

“Saturated 7 to 12 membered heterobicyclyl” or “saturated 7 to 12 membered heterobicycle” means fully saturated 7 to 12 membered heterobicyclyl or 7 to 12 membered heterobicycle.

“7 to 12 membered at least partly saturated heterobicyclyl” or “7 to 12 membered at least partly saturated heterobicycle” means an at least partly saturated “7 to 12 membered heterobicyclyl” or “7 to 12 membered heterobicycle”.

“9 to 11 membered aromatic heterobicyclyl” or “9 to 11 membered aromatic heterobicycle” means a heterocyclic system of two rings, wherein at least one ring is aromatic and wherein the heterocyclic ring system has 9 to 11 ring atoms, where two ring atoms are shared by both rings and that may contain up to the maximum number of double bonds (fully or partially aromatic) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 9 to 11 membered aromatic heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, dihydroquinoline, tetrahydroquinoline, isoquinoline, tetrahydroisoquinoline, dihydroisoquinoline, benzazepine, purine or pteridine. The terms “9 to 10 membered aromatic heterobicyclyl” or “9 to 10 membered aromatic heterobicycle” are defined accordingly.

Preferred compounds of formula (I) are those compounds in which one or more of the residues contained therein have the meanings given below, with all combinations of preferred substituent definitions being a subject of the present invention. With respect to all preferred compounds of the formula (I) the present invention also includes all tautomeric and stereoisomeric forms and mixtures thereof in all ratios, and their pharmaceutically acceptable salts.

In preferred embodiments of the present invention, the substituents mentioned below independently have the following meaning. Hence, one or more of these substituents can have the preferred or more preferred meanings given below.

Preferably, A¹ is a nitrogen ring atom containing 5-membered heterocyclene, wherein A¹ is optionally substituted with one or more R⁴, which are the same or different.

Preferably, A¹ is a nitrogen ring atom containing 5-membered heterocyclene selected from the group of bivalent heterocycles consisting of oxadiazole, imidazole, imidazolidine, pyrazole and triazole, preferably oxadiazole, and wherein A¹ is optionally substituted with one or more R⁴, which are the same or different.

Preferably, A¹ is unsubstituted or substituted with one or two R⁴, which are the same or different, preferably A¹ is unsubstituted.

Preferably, R⁴ is oxo, where the ring is at least partially saturated.

Preferably, A¹ is

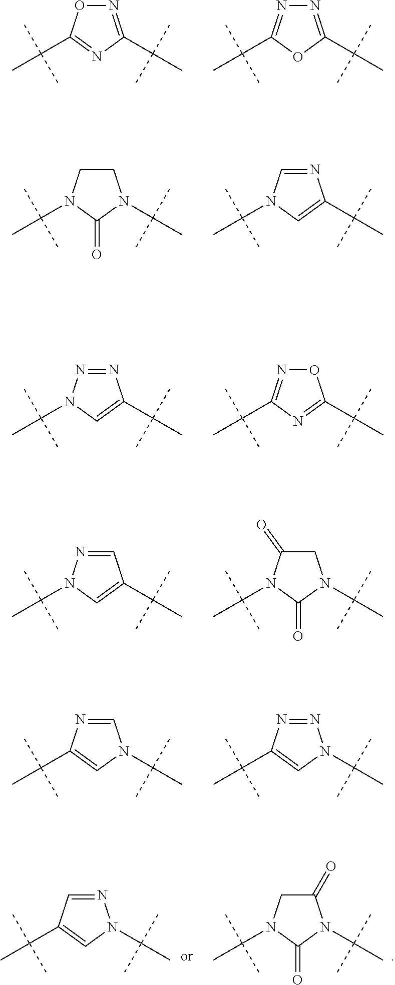

More preferably, A¹ is

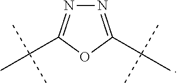

Preferably, A² is phenyl, pyridyl, pyrazinyl, pyridazinyl, pyrazolyl or 1,2,4-oxadiazolyl, wherein A² is optionally substituted with one or more R⁶, which are the same or different.

Preferably, A² is phenyl, pyridyl, pyrazinyl or pyridazinyl, wherein A² is optionally substituted with one or more R⁶, which are the same or different.

Preferably, A² is substituted with one or two R⁶, which are the same or different.

Preferably, each R⁶ is independently F, Cl, CF₃, OCH₃, CH₃, CH₂CH₃, or cyclopropyl.

Preferably, R² is H.

Preferably, R³ is A³.

Preferably, A³ is phenyl, pyridyl, pyrazinyl or pyrimidazyl, wherein A³ is optionally substituted with one or more R⁸, which are the same or different.

Preferably, A³ is substituted with one or two R⁸, which are the same or different.

Preferably, R² and R³ are joined to form a dihydrobenzopyran ring, wherein the ring is optionally substituted with one or more R⁸, which are the same or different, preferably the ring is substituted with one or two R⁸. Accordingly, a preferred formula (I) is formula (Ia)

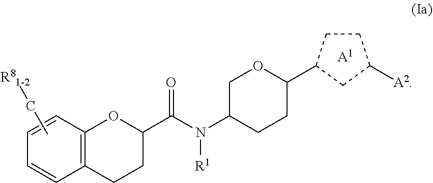

However in another preferred embodiment R³ is A³.

Preferably, R⁸ is independently F, Cl, CF₃, CH═O, CH₂OH or CH₃.

Compounds of the formula (I) in which some or all of the above-mentioned groups have the preferred or more preferred meanings are also an object of the present invention.

Preferred specific compounds of the present invention are selected from the group consisting of
2-(4-chloro-3-fluorophenoxy)-N-[(3R,6S)-6-[5-(4-chlorophenyl)-1,3,4-oxadiazol-2-yl]oxan-3-yl]acetamide,
2-(4-chlorophenoxy)-N-[(3R,6S)-6-[5-(4-chlorophenyl)-1,3,4-oxadiazol-2-yl]oxan-3-yl]acetamide,
2-(4-chloro-3-fluorophenoxy)-N-[(3R,6S)-6-{5-[6-(trifluoromethyl)pyridin-3-yl]-1,3,4-oxadiazol-2-yl}oxan-3-yl]acetamide,
2-(4-chloro-3-fluorophenoxy)-N-[(3S,6R)-6-[5-(4-chlorophenyl)-1,3,4-oxadiazol-2-yl]oxan-3-yl]acetamide,
2-(4-chloro-3-fluorophenoxy)-N-[(3R,6S)-6-[5-(6-cyclopropylpyridin-3-yl)-1,3,4-oxadiazol-2-yl]oxan-3-yl]acetamide,
2-(4-chloro-3-fluorophenoxy)-N-[(3R,6S)-6-[5-(6-ethylpyridin-3-yl)-1,3,4-oxadiazol-2-yl]oxan-3-yl]acetamide,
2-[(6-chloro-5-fluoropyridin-3-yl)oxy]-N-[(3R,6S)-6-[5-(4-chlorophenyl)-1,3,4-oxadiazol-2-yl]oxan-3-yl]acetamide,
N-[(3R,6S)-6-[5-(4-chlorophenyl)-1,3,4-oxadiazol-2-yl]oxan-3-yl]-2-{[2-(trifluoromethyl)pyridin-4-yl]oxy}acetamide,
N-[(3R,6S)-6-[5-(4-chlorophenyl)-1,3,4-oxadiazol-2-yl]oxan-3-yl]-2-[(6-chloropyridin-3-yl)oxy]acetamide,
N-[(3R,6S)-6-[5-(4-chlorophenyl)-1,3,4-oxadiazol-2-yl]oxan-3-yl]-2-[(5-fluoro-6-methylpyridin-3-yl)oxy]acetamide,
2-[(6-chloro-5-fluoropyridin-3-yl)oxy]-N-[(3R,6S)-6-[5-(6-chloropyridin-3-yl)-1,3,4-oxadiazol-2-yl]oxan-3-yl]acetamide,
N-[(3R,6S)-6-[5-(4-chlorophenyl)-1,3,4-oxadiazol-2-yl]oxan-3-yl]-2-[(6-methylpyridin-3-yl)oxy]acetamide,
N-[(3R,6S)-6-[5-(4-chlorophenyl)-1,3,4-oxadiazol-2-yl]oxan-3-yl]-2-[(5-chloropyrazin-2-yl)oxy]acetamide,
N-[(3R,6S)-6-[5-(4-chlorophenyl)-1,3,4-oxadiazol-2-yl]oxan-3-yl]-2-[(2-chloropyrimidin-5-yl)oxy]acetamide,
2-[(5-chloro-6-methylpyridin-3-yl)oxy]-N-[(3R,6S)-6-[5-(4-chlorophenyl)-1,3,4-oxadiazol-2-yl]oxan-3-yl]acetamide,
2-(4-chloro-3-fluorophenoxy)-N-[(3R,6S)-6-{5-[5-(trifluoromethyl)pyridin-3-yl]-1,3,4-oxadiazol-2-yl}oxan-3-yl]acetamide,
2-(4-chloro-3-fluorophenoxy)-N-[(3R,6S)-6-{5-[2-(trifluoromethyl)pyridin-4-yl]-1,3,4-oxadiazol-2-yl}oxan-3-yl]acetamide,
N-[3R,6S)-6-[5-(4-chlorophenyl)-1,3,4-oxadiazol-2-yl]tetrahydropyran-3-yl]-2-[[6-(trifluoromethyl)-3-pyridyl]oxy]acetamide, or
N-[(3R,6S)-6-[5-(4-chlorophenyl)-1,3,4-oxadiazol-2-yl]oxan-3-yl]-2-{[5-(trifluoromethyl)pyridin-3-yl]oxy}acetamide.

Where tautomerism, like e.g. keto-enol tautomerism, of compounds of formula (I) may occur, the individual forms, like e.g. the keto and enol form, are comprised separately and together as mixtures in any ratio. Same applies to stereoisomers, like e.g. enantiomers, cis/trans isomers, conformers and the like.

Especially, when enantiomeric or diastereomeric forms are given in a compound according to formula (I) each pure form separately and any mixture of at least two of the pure forms in any ratio is comprised by formula (I) and is a subject of the present invention.

A preferred formula (I) is formula (Ib)

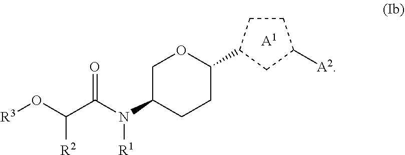

Isotopic labeled compounds of formula (I) are also within the scope of the present invention. Methods for isotope labeling are known in the art. Preferred isotopes are those of the elements H, C, N, O and S. Solvates and hydrates of compounds of formula (I) are also within the scope of the present invention.

If desired, isomers can be separated by methods well known in the art, e.g. by liquid chromatography. Same applies for enantiomers by using e.g. chiral stationary phases.

Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) may be obtained from stereoselective synthesis using optically pure starting materials, reagents and/or catalysts.

In case the compounds according to formula (I) contain one or more acidic or basic groups, the invention also comprises their corresponding pharmaceutically or toxicologically acceptable salts, in particular their pharmaceutically utilizable salts. Thus, the compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) which, owing to low physiological compatibility, are not directly suitable for use in pharmaceuticals but which can be used, for example, as intermediates for chemical reactions or for the preparation of pharmaceutically acceptable salts.

As shown below compounds of the present invention are believed to be suitable for modulating the integrated stress response pathway.

The Integrated Stress Response (ISR) is a cellular stress response common to all eukaryotes (1). Dysregulation of ISR signaling has important pathological consequences linked inter alia to inflammation, viral infection, diabetes, cancer and neurodegenerative diseases. ISR is a common denominator of different types of cellular stresses resulting in phosphorylation of the alpha subunit of eukaryotic translation initiation factor 2 (eIF2alpha) on serine 51 leading to the suppression of normal protein synthesis and expression of stress response genes (2). In mammalian cells the phosphorylation is carried out by a family of four eIF2alpha kinases, namely: PKR-like ER kinase (PERK), double-stranded RNA-dependent protein kinase (PKR), heme-regulated eIF2alpha kinase (HRI), and general control nonderepressible 2 (GCN2), each responding to distinct environmental and physiological stresses (3).

eIF2alpha together with eIF2beta and eIF2gamma form the eIF2 complex, a key player of the initiation of normal mRNA translation (4). The eIF2 complex binds GTP and Met-tRNAi forming a ternary complex (eIF2-GTP-Met-tRNAi), which is recruited by ribosomes for translation initiation (5, 6).

eIF2B is a heterodecameric complex consisting of 5 subunits (alpha, beta, gamma, delta, epsilon) which in duplicate form a GEF-active decamer (7).

In response to ISR activation, phosphorylated eIF2alpha inhibits the eIF2B-mediated exchange of GDP for GTP, resulting in reduced ternary complex formation and hence in the inhibition of translation of normal mRNAs characterized by ribosomes binding to the 5′ AUG start codon (8). Under these conditions of reduced ternary complex abundance the translation of several specific mRNAs including the mRNA coding for the transcription factor ATF4 is activated via a mechanism involving altered translation of upstream ORFs (uORFs) (7, 9, 10). These mRNAs typically contain one or more uORFs that normally function in unstressed cells to limit the flow of ribosomes to the main coding ORF. For example, during normal conditions, uORFs in the 5′ UTR of ATF occupy the ribosomes and prevent translation of the coding sequence of ATF4. However, during stress conditions, i.e. under conditions of reduced ternary complex formation, the probability for ribosomes to scan past these upstream ORFs and initiate translation at the ATF4 coding ORF is increased. ATF4 and other stress response factors expressed in this way subsequently govern the expression of an array of further stress response genes. The acute phase consists in expression of proteins that aim to restore homeostasis, while the chronic phase leads to expression of pro-apoptotic factors (1, 11, 12, 13).

Upregulation of markers of ISR signaling has been demonstrated in a variety of conditions, among these cancer and neurodegenerative diseases. In cancer, ER stress-regulated translation increases tolerance to hypoxic conditions and promotes tumor growth (14, 15, 16), and deletion of PERK by gene targeting has been shown to slow growth of tumours derived from transformed PERK^{-/-} mouse embryonic fibroblasts (14, 17). Further, a recent report has provided proof of concept using patient derived xenograft modeling in mice for activators of eIF2B to be effective in treating a form of aggressive metastatic prostate cancer (28). Taken together, prevention of cytoprotective ISR signaling may represent an effective antiproliferation strategy for the treatment of at least some forms of cancer.

Further, modulation of ISR signaling could prove effective in preserving synaptic function and reducing neuronal decline, also in neurodegenerative diseases that are characterized by misfolded proteins and activation of the unfolded protein response (UPR), such as amyotrophic lateral sclerosis (ALS), frontotemporal dementia (FTD), Alzheimer's disease (AD), Parkinson's disease (PD) and Jakob Creutzfeld (prion) diseases (18, 19, 20). With prion disease an example of a neurodegenerative disease exists where it has been shown that pharmacological as well as genetic inhibition of ISR signaling can normalize protein translation levels, rescue synaptic function and prevent neuronal loss (21). Specifically, reduction of levels of phosphorylated eIF2alpha by overexpression of the phosphatase controlling phosphorylated eIF2alpha levels increased survival of prion-infected mice whereas sustained eIF2alpha phosphorylation decreased survival (22).

Further, direct evidence for the importance of control of protein expression levels for proper brain function exists in the form of rare genetic diseases affecting functions of eIF2 and eIF2B. A mutation in eIF2gamma that disrupts complex integrity of eIF2 and hence results in reduced normal protein expression levels is linked to intellectual disability syndrome (ID) (23). Partial loss of function mutations in subunits of eIF2B have been shown to be causal for the rare leukodystrophy Vanishing White Matter Disease (VWMD) (24, 25). Specifically, stabilization of eIF2B partial loss of function in a VWMD mouse model by a small molecule related to ISRIB has been shown to reduce ISR markers and improve functional as well as pathological end points (26, 27).

The present invention provides compounds of the present invention in free or pharmaceutically acceptable salt form to be used in the treatment of diseases or disorders mentioned herein.

Thus a further aspect of the present invention is a compound or a pharmaceutically acceptable salt thereof of the present invention for use as a medicament.

The therapeutic method described may be applied to mammals such as dogs, cats, cows, horses, rabbits, monkeys and humans. Preferably, the mammalian patient is a human patient.

Accordingly, the present invention provides a compound or a pharmaceutically acceptable salt thereof of the present invention to be used in the treatment or prevention of one or more diseases or disorders associated with integrated stress response.

A further aspect of the present invention is a compound or a pharmaceutically acceptable salt thereof of the present invention for use in a method of treating or preventing one or more disorders or diseases associated with integrated stress response.

A further aspect of the present invention is the use of a compound or a pharmaceutically acceptable salt thereof of the present invention for the manufacture of a medicament for the treatment or prophylaxis of one or more disorders or diseases associated with integrated stress response.

Yet another aspect of the present invention is a method for treating, controlling, delaying or preventing in a mammalian patient in need of the treatment of one or more diseases or disorders associated with integrated stress response, wherein the method comprises administering to said patient a therapeutically effective amount of a compound or a pharmaceutically acceptable salt thereof of the present invention.

The present invention provides a compound or a pharmaceutically acceptable salt thereof of the present invention to be used in the treatment or prevention of one or more diseases or disorders mentioned below.

A further aspect of the present invention is a compound or a pharmaceutically acceptable salt thereof of the present invention for use in a method of treating or preventing one or more disorders or diseases mentioned below.

A further aspect of the present invention is the use of a compound or a pharmaceutically acceptable salt thereof of the present invention for the manufacture of a medicament for the treatment or prophylaxis of one or more disorders or diseases mentioned below.

Yet another aspect of the present invention is a method for treating, controlling, delaying or preventing in a mammalian patient in need of the treatment of one or more diseases or disorders mentioned below, wherein the method comprises administering to said patient a therapeutically effective amount of a compound or a pharmaceutically acceptable salt thereof of the present invention.

Diseases or disorders include but are not limited to leukodystrophies, intellectual disability syndrome, neurodegenerative diseases and disorders, neoplastic diseases, infectious diseases, inflammatory diseases, musculoskeletal diseases, metabolic diseases, ocular diseases as well as diseases selected from the group consisting of organ fibrosis, chronic and acute diseases of the liver, chronic and acute diseases of the lung, chronic and acute diseases of the kidney, myocardial infarction, cardiovascular disease, arrhythmias, atherosclerosis, spinal cord injury, ischemic stroke, and neuropathic pain.

Leukodystrophies

Examples of leukodystrophies include, but are not limited to, Vanishing White Matter Disease (VWMD) and childhood ataxia with CNS hypo-myelination (e.g. associated with impaired function of eIF2 or components in a signal transduction or signaling pathway including eIF2).

Intellectual Disability Syndrome

Intellectual disability in particular refers to a condition in which a person has certain limitations in intellectual functions like communicating, taking care of him- or herself, and/or has impaired social skills. Intellectual disability syndromes include, but are not limited to, intellectual disability conditions associated with impaired function of eIF2 or components in a signal transduction or signaling pathway including eIF2.

Neurodegenerative Diseases/Disorders

Examples of neurodegenerative diseases and disorders include, but are not limited to, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, Kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Progressive supranuclear palsy, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, and tauopathies.

In particular, the neurodegenerative disease or and disorder is selected from the group consisting of Alzheimer's disease, Parkinson's disease and amyotrophic lateral sclerosis.

Neoplastic Diseases

A neoplastic disease may be understood in the broadest sense as any tissue resulting from miss-controlled cell growth. In many cases a neoplasm leads to at least bulky tissue mass optionally innervated by blood vessels. It may or may not comprise the formation of one or more metastasis/metastases. A neoplastic disease of the present invention may be any neoplasm as classified by the International Statistical Classification of Diseases and Related Health Problems 10th Revision (ICD-10) classes C00-D48.

Exemplarily, a neoplastic disease according to the present invention may be the presence of one or more malignant neoplasm(s) (tumors) (ICD-10 classes C00-C97), may be the presence of one or more in situ neoplasm(s) (ICD-10 classes D00-D09), may be the presence of one or more benign neoplasm(s) (ICD-10 classes D10-D36), or may be the presence of one or more neoplasm(s) of uncertain or unknown behavior (ICD-10 classes D37-D48). Preferably, a neoplastic disease according to the present invention refers to the presence of one or more malignant neoplasm(s), i.e., is malignant neoplasia (ICD-10 classes C00-C97).

In a more preferred embodiment, the neoplastic disease is cancer.

Cancer may be understood in the broadest sense as any malignant neoplastic disease, i.e., the presence of one or more malignant neoplasm(s) in the patient. Cancer may be solid or hematologic malignancy. Contemplated herein are without limitation leukemia, lymphoma, carcinomas and sarcomas.

In particular, neoplastic diseases, such as cancers, characterized by upregulated ISR markers are included herein.

Exemplary cancers include, but are not limited to, thyroid cancer, cancers of the endocrine system, pancreatic cancer, brain cancer (e.g. glioblastoma multiforme, glioma), breast cancer (e.g. ER positive, ER negative, chemotherapy resistant, herceptin resistant, HER2 positive, doxorubicin resistant, tamoxifen resistant, ductal carcinoma, lobular carcinoma, primary, metastatic), cervix cancer, ovarian cancer, uterus cancer, colon cancer, head & neck cancer, liver cancer (e.g. hepatocellular carcinoma), kidney cancer, lung cancer (e.g. non-small cell lung carcinoma, squamous cell lung carcinoma, adenocarcinoma, large cell lung carcinoma, small cell lung carcinoma, carcinoid, sarcoma), colon cancer, esophageal cancer, stomach cancer, bladder cancer, bone cancer, gastric cancer, prostate cancer and skin cancer (e.g. melanoma).

Further examples include, but are not limited to, myeloma, leukemia, mesothelioma, and sarcoma.

Additional examples include, but are not limited to, Medulloblastoma, Hodgkin's Disease, Non-Hodgkin's Lymphoma, multiple myeloma, neuroblastoma, glioma, glioblastoma multiforme, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, and cancer of the hepatic stellate cells.

Exemplary leukemias include, but are not limited to, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

Exemplary sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Exemplary melanomas include, but are not limited to, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

Exemplary carcinomas include, but are not limited to, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, and carcinoma villosum.

Infectious diseases Examples include, but are not limited to, infections caused by viruses (such as infections by HIV-1: human immunodeficiency virus type 1; IAV: influenza A virus; HCV: hepatitis C virus; DENV: dengue virus; ASFV: African swine fever virus; EBV: Epstein-Barr virus; HSV1: herpes simplex virus 1; CHIKV: chikungunya virus; HCMV: human cytomegalovirus; SARS-CoV: severe acute respiratory syndrome coronavirus); SARS-CoV-2: severe acute respiratory syndrome coronavirus 2) and infections caused by bacteria (such as infections by Legionella, Brucella, Simkania, Chlamydia, Helicobacter and Campylobacter).

Inflammatory Diseases

Examples of inflammatory diseases include, but are not limited to, postoperative cognitive dysfunction (decline in cognitive function after surgery), traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

Musculoskeletal Diseases

Examples of musculoskeletal diseases include, but are not limited to, muscular dystrophy, multiple sclerosis, Freidrich's ataxia, a muscle wasting disorder (e.g., muscle atrophy, sarcopenia, cachexia), inclusion body myopathy, progressive muscular atrophy, motor neuron disease, carpal tunnel syndrome, epicondylitis, tendinitis, back pain, muscle pain, muscle soreness, repetitive strain disorders, and paralysis.

Metabolic Diseases

Examples of metabolic diseases include, but are not limited to, diabetes (in particular diabetes Type II), non-alcoholic steatohepatitis (NASH), non-alcoholic fatty liver disease (NAFLD), Niemann-Pick disease, liver fibrosis, obesity, heart disease, atherosclerosis, arthritis, cystinosis, phenylketonuria, proliferative retinopathy, and Kearns-Sayre disease.

Ocular Diseases

Examples of ocular diseases include, but are not limited to, edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

Further Diseases

Further diseases include, but are not limited to, organ fibrosis (such as liver fibrosis, lung fibrosis, or kidney fibrosis), chronic and acute diseases of the liver (such as fatty liver disease, or liver steatosis), chronic and acute diseases of the lung, chronic and acute diseases of the kidney, myocardial infarction, cardiovascular disease, arrhythmias, atherosclerosis, spinal cord injury, ischemic stroke, and neuropathic pain.

Yet another aspect of the present invention is a pharmaceutical composition comprising at least one compound or a pharmaceutically acceptable salt thereof of the present invention together with a pharmaceutically acceptable carrier, optionally in combination with one or more other bioactive compounds or pharmaceutical compositions.

Preferably, the one or more bioactive compounds are modulators of the integrated stress response pathway other than compounds of formula (I).

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

A pharmaceutical composition of the present invention may comprise one or more additional compounds as active ingredients like a mixture of compounds of formula (I) in the composition or other modulators of the integrated stress response pathway.

The active ingredients may be comprised in one or more different pharmaceutical compositions (combination of pharmaceutical compositions).

The term “pharmaceutically acceptable salts” refers to salts prepared from pharmaceutically acceptable non-toxic bases or acids, including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally, for example, as liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula (I) may also be administered parenterally. Solutions or suspensions of these active compounds can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) are administered orally.

The effective dosage of active ingredient employed may vary depending on the particular compound employed, the mode of administration, the condition being treated and the severity of the condition being treated. Such dosage may be ascertained readily by a person skilled in the art.

Starting materials for the synthesis of preferred embodiments of the invention may be purchased from commercially available sources such as Array, Sigma Aldrich, Acros, Fisher, Fluka, ABCR or can be synthesized using known methods by one skilled in the art.

In general, several methods are applicable to prepare compounds of the present invention. In some cases various strategies can be combined. Sequential or convergent routes may be used. Exemplary synthetic routes are described below.

### EXAMPLES

### I Chemical Synthesis

### Experimental Procedures

The following Abbreviations and Acronyms are used:
aq aqueous
Brine saturated solution of NaCl in water
CV column volume
δ chemical shifts in parts per million
d doublet
DCM dichloromethane
dd doublet of doublet
ddd doublet of doublet of doublet
DMSO dimethylsulfoxide
DMSO-dδ deuterated dimethylsulfoxide
DIPEA diisopropylethylamine
DMF dimethyl formamide
ESI+ positive ionisation mode
ESI− negative ionisation mode
EtOAc ethyl acetate
Et₂O diethyl ether
HCl Hydrochloric acid
HPLC High-performance liquid chromatography
h hour(s)
J NMR coupling constant
MgSO₄ Magnesium sulphate
m multiplet
mL millilitre (s)
min minutes
N₂ nitrogen atmosphere
Na₂SO₄ sodium sulphate
NaHCO₃ sodium bicarbonate
NaOH sodium hydroxide
NMR Nuclear Magnetic Resonance
q Quintuplet
r.t. Room temperature
RT Retention time
s singlet
t triplet
TBME tert-butyl-methylether
THE tetrahydrofuran
HATU 1-[Bis(dimethylamino)methylidene]-1H-[1,2,3]triazolo[4,5-b]pyridin-1-ium-3-oxide hexa fluorophosphate

Analytical LCMS conditions are as follows:

System 1 (S1): ACIDIC IPC METHOD (MS17):

Analytical METCR1410 HPLC-MS were performed on Shimadzu LCMS-2010EV systems using a reverse phase Kinetex Core shell C18 columns (2.1 mm×50 mm, 5 μm; temperature: 40° C.) and a gradient of 5-100% B (A=0.1% formic acid in water; B=0.1% formic acid in acetonitrile) over 1.2 min then 100% B for 0.1 min, with an injection volume of 3 μL at flow rate of 1.2 mL/min. UV spectra were recorded at 215 nm using a SPD-M20A photo diode array detector. Mass spectra were obtained over the range m/z 150 to 850 at a sampling rate of 2 scans per sec using a LCMS2010EV. Data were integrated and reported using Shimadzu LCMS-Solutions and PsiPort software.

System 2 (S2): ACIDIC FINAL METHOD (MSQ1 and MSQ2):

System 2A: Analytical MET-uHPLC-AB-101 HPLC-MS were performed on a Waters Acquity uPLC system with Waters PDA and ELS detectors using a Phenomenex Kinetex-XB C18 column (2.1 mm×100 mm, 1.7 μM; temperature: 40° C.) and a gradient of 5-100% B (A=0.1% formic acid in water; B=0.1% formic acid in acetonitrile) over 5.3 min then 100% B for 0.5 min, with an injection solution of 3 μL at flow rate of 0.6 mL/min. UV spectra were recorded at 215 nm using a Waters Acquity photo diode array detector. Mass spectra were obtained over the range m/z 150 to 850 at a sampling rate of 5 scans per sec using a Waters SQD. Data were integrated and reported using Waters MassLynx and OpenLynx software.

System 2B: Analytical MET-uHPLC-AB-102 HPLC-MS were performed on a Waters Acquity uPLC system with Waters PDA and ELS detectors using a Waters uPLC CSH C18 column (2.1 mm×100 mm, 1.7 μM; temperature: 40° C.) and a gradient of 5-100% (A=2 mM ammonium bicarbonate, buffered to pH 10 with ammonium hydroxide solution; B=acetonitrile) over 5.3 min then 100% B for 0.5 min at flow rate of 0.6 mL/min. UV spectra were recorded at 215 nm using a Waters Acquity photo diode array detector. Mass spectra were obtained over the range m/z 150 to 850 at a sampling rate of 5 scans per sec using a Waters Quatro Premier XE. Data were integrated and reported using Waters MassLynx and OpenLynx software.

System 3 (S3): ACIDIC FINAL METHOD (Shimadzu):

5% Solvent B for 1 min and then Linear gradient 5-100% solvent B in 5.5 mins+2.5 mins 100% solvent B at flow rate 1.0 ml/min. Column ATLANTIS dC18 (50×3.0 mm). Solvent A=0.1% Formic acid in water, Solvent B=0.1% Formic acid in Acetonitrile

System 4 (S4): BASIC FINAL METHOD (MS16)

Analytical METCR1603 HPLC-MS were performed on a Agilent G1312A system with Waters 2996 PDA detector and Waters 2420 ELS detector using a Phenomenex Gemini—NX C18 column (2.0×100 mm, 3 mm column; temperature: 40° C.) and a gradient of 5-100% (A=2 mM ammonium bicarbonate, buffered to pH 10; B=acetonitrile) over 5.5 min then 100% B for 0.4 min, with an injection volume of 3 μL and at flow rate of 0.6 mL/min. UV spectra were recorded at 215 nm using a Waters Acquity photo diode array detector. Mass spectra were obtained over the range m/z 150 to 850 at a sampling rate of 5 scans per sec using a Waters ZQ mass detector. Data were integrated and reported using Waters MassLynx and OpenLynx software.

Preparative HPLC conditions are as follows:

Method 1: Reverse phase chromatography using acidic pH, standard elution method Purifications by FCC on reverse phase silica (acidic pH, standard elution method) were performed on Biotage Isolera systems using the appropriate SNAP C18 cartridge and a gradient of 10% B (A=0.1% formic acid in water; B=0.1% formic acid in acetonitrile) over 1.7 CV then 10-100% B over 19.5 CV and 100% B for 2 CV.

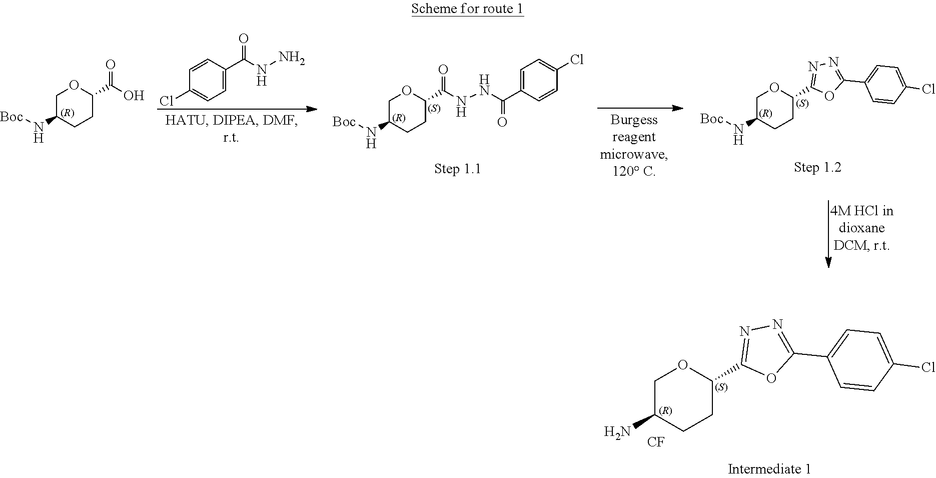

### Intermediate 1: [(3R,6S)-6-[5-(4-chlorophenyl)-1,3,4-oxadiazol-2-yl]tetrahydropyran-3-yl]ammonium chloride

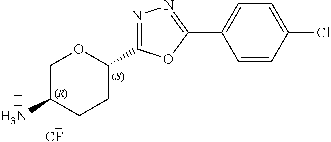

To a solution of tert-butyl N-[(3R,6S)-6-[5-(4-chlorophenyl)-1,3,4-oxadiazol-2-yl]tetrahydropyran-3-yl]carbamate (90%, 227 mg, 0.539 mmol) in DCM (1.35 mL) was added a solution of 4 M HCl in Dioxane (1.4 mL, 5.40 mmol) at r.t. and the reaction stirred at this temperature for 1 h. The solvent was removed under reduced pressure to afford[(3R,6S)-6-[5-(4-chlorophenyl)-1,3,4-oxadiazol-2-yl]tetrahydropyran-3-yl]ammonium chloride (199 mg, 0.522 mmol, 97% yield) as an off-white powder. ¹H NMR (500 MHz, DMSO-d₆) δ 8.17 (s, 3H), 8.08-7.96 (m, 2H), 7.75-7.64 (m, 2H), 4.90 (dd, J=10.2, 2.5 Hz, 1H), 4.09 (dd, J=10.8, 3.5 Hz, 1H), 3.59-3.54 (m, 1H), 3.29-3.26 (m, 1H), 2.27-2.17 (m, 2H), 2.09-1.97 (m, 1H), 1.83-1.70 (m, 1H). M/Z: 280, 282 [M+H], ESI+, RT=2.46 min (S4).

### Step 1.1: tert-butyl N-[(3R,6S)-6-[[(4-chlorobenzoyl)amino]carbamoyl]tetrahydropyran-3-yl]carbamate

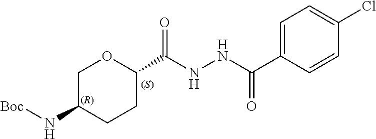

HATU (651 mg, 1.71 mmol) was added to a solution of 4-chlorobenzohydrazide (243 mg, 1.43 mmol) and DIPEA (0.75 mL, 4.28 mmol) in dry DMF (4 mL) at r.t. and stirred for 10 min. (2S,5R)-5-(tert-butoxycarbonylamino)tetrahydropyran-2-carboxylic acid (350 mg, 1.43 mmol) was then added and the reaction mixture was stirred at r.t. for 2 h. The reaction mixture was diluted with water (30 mL) and Et₂O (30 mL), causing a tan solid to precipitate. The solid was filtered, washed with Et₂O, and the residual solvent was removed in vacuo to give tert-butyl N-[(3R,6S)-6-[[(4-chlorobenzoyl)amino]carbamoyl]tetrahydropyran-3-yl]carbamate (522 mg, 1.25 mmol, 87% Yield) as a tan solid. ¹H NMR (400 MHz, DMSO-d6) δ 10.38 (s, 1H), 9.76 (s, 1H), 7.88 (d, J=8.6 Hz, 2H), 7.57 (d, J=8.6 Hz, 2H), 6.84 (d, J=7.9 Hz, 1H), 3.91 (d, J=7.3 Hz, 1H), 3.87-3.74 (m, 1H), 3.38 (d, J=7.0 Hz, 1H), 3.06 (t, J=10.6 Hz, 1H), 1.94 (t, J=13.2 Hz, 2H), 1.62-1.43 (m, 2H), 1.39 (s, 9H). M/Z: 342, 344 [M-tBu+H], ESI+, RT=1.21 min (S1).

### Step 1.2: tert-butyl N-[(3R,6S)-6-[5-(4-chlorophenyl)-1,3,4-oxadiazol-2-yl]tetrahydropyran-3-yl]carbamate

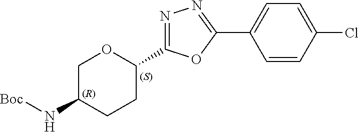

A suspension of tert-butyl N-[(3R,6S)-6-[[(4-chlorobenzoyl)amino]carbamoyl]tetrahydropyran-3-yl]carbamate (372 mg, 0.673 mmol) and methoxycarbonyl-(triethylammonio)sulfonyl-azanide (642 mg, 2.69 mmol) in dry THE (4 mL) was stirred at 120° C. for 10 min under microwave irradiation (normal absorption). The resultant solution was partitioned between water (25 mL) and EtOAc (25 mL), with the organic layer washed with brine (25 mL), dried (MgSO₄), filtered and concentrated in vacuo. The residual material was purified using flash chromatography on silica, eluting with heptanes-EtOAc, 1:0 to 0:1 to afford tert-butyl N-[(3R,6S)-6-[5-(4-chlorophenyl)-1,3,4-oxadiazol-2-yl]tetrahydropyran-3-yl]carbamate (227 mg, 0.539 mmol, 80% Yield) as an off-white powder. ¹H NMR (500 MHz, Chloroform-d) δ 8.04-7.97 (m, 2H), 7.52-7.45 (m, 2H), 4.72 (dd, J=9.6, 3.0 Hz, 1H), 4.48 (s, 1H), 4.23-4.14 (m, 1H), 3.82-3.72 (m, 1H), 3.30 (t, J=10.2 Hz, 1H), 2.32-2.10 (m, 2H), 1.58 (d, J=18.1 Hz, 2H), 1.46 (s, 9H). M/Z: 324, 326 [M-tBu+H], ESI+, RT=1.21 min (S1).

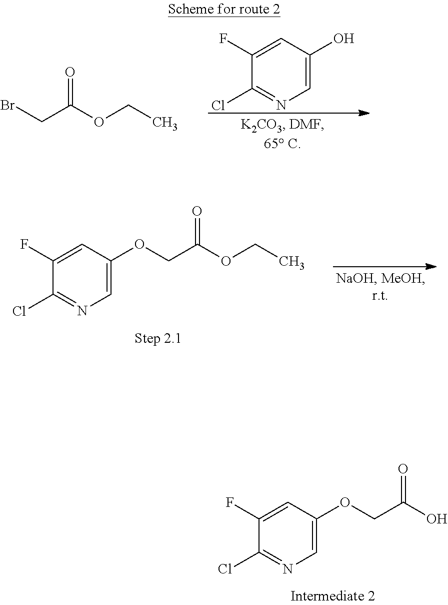

### Intermediate 2: 2-[(6-chloro-5-fluoro-3-pyridyl)oxy]acetic acid

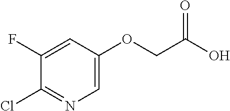 [SMILES: O=C(O)COc1cnc(Cl)c(F)c1]

An aqueous solution of 2 M NaOH (12 mL, 24.7 mmol) was added to a solution of ethyl 2-[(6-chloro-5-fluoro-3-pyridyl)oxy]acetate (96%, 6.01 g, 24.7 mmol) in methanol (15 mL) at r.t. and stirred for 2 h. The reaction mixture was concentrated and then acidified to pH 4 with 1 N HCl solution. The precipitated solid was filtered to give 2-[(6-chloro-5-fluoro-3-pyridyl)oxy]acetic acid (1.00 g, 4.67 mmol, 19% Yield) as a beige solid. ¹H NMR (500 MHz, DMSO-d₆) δ 8.06 (d, J=2.6 Hz, 1H), 7.73 (dd, J=10.4, 2.6 Hz, 1H), 4.82 (s, 2H). M/Z: 206, 208, ESI+, RT=0.85 min (S1).

### Step 2.1: ethyl 2-[(6-chloro-5-fluoro-3-pyridyl)oxy]acetate

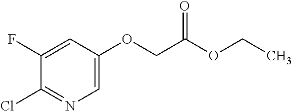

Ethyl 2-bromoacetate (3.4 mL, 30.2 mmol) was added to a suspension of 6-chloro-5-fluoropyridin-3-ol (4.25 g, 28.8 mmol) and potassium carbonate (11.94 g, 86.4 mmol) in DMF (12 mL) and stirred at 65° C. for 1 h and allowed to cool to r.t. and to stand overnight at r.t. The reaction mixture was suspended in EtOAc (20 mL) and filtered. The filtrate was washed with water (50 mL), brine (50 mL), dried over Na₂SO₄, filtered and evaporated to afford ethyl 2-[(6-chloro-5-fluoro-3-pyridyl)oxy]acetate (6.01 g, 24.7 mmol, 86% Yield) as a green solid. ¹H NMR (500 MHz, Chloroform-d) δ 7.92 (d, J=2.6 Hz, 1H), 7.08 (dd, J=9.1, 2.6 Hz, 1H), 4.65 (s, 2H), 4.26 (q, J=7.1 Hz, 2H), 1.29 (t, J=7.1 Hz, 3H). M/Z: 234, 236 [M+1], ESI+, RT=1.09 min (S1).

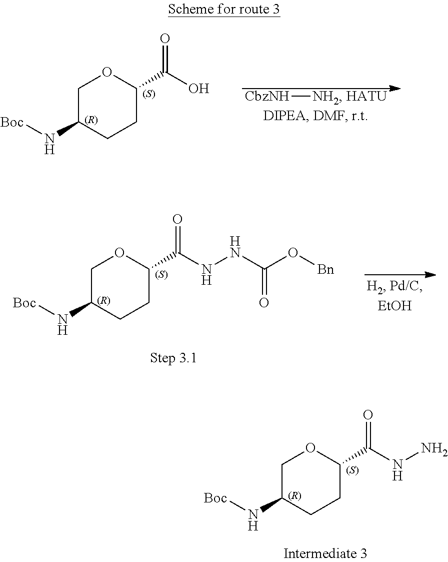

### Intermediate 3: tert-butyl N-[3R,6S)-6-(hydrazinecarbonyl)tetrahydropyran-3-yl]carbamate

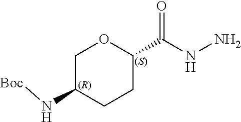

To a degassed solution of tert-butyl N-[(3R,6S)-6-(benzyloxycarbonylaminocarbamoyl)tetrahydropyran-3-yl]carbamate (950 mg, 2.41 mmol) in Ethanol (25 mL) and EtOAc (15 mL) at r.t. was added palladium on charcoal (10%, 95 mg, 0.089 mmol) and the reaction mixture stirred under an atmosphere of hydrogen for 3 h. The reaction was stopped by switching the atmosphere to N₂. The reaction mixture was warmed to near reflux and filtered hot through a pad of Celite®, washing copiously with ethanol. The filtrates were concentrated to dryness to afford tert-butyl N-[(3R,6S)-6-(hydrazinecarbonyl)tetrahydropyran-3-yl]carbamate (678 mg, 2.46 mmol, 100% Yield) as an off-white powder. ¹H NMR (500 MHz, DMSO-d₆) δ 8.86 (s, 1H), 6.80 (d, J=7.7 Hz, 1H), 4.20 (s, 2H), 3.91-3.80 (m, 1H), 3.68-3.62 (m, 1H), 3.02-2.94 (m, 1H), 1.93-1.82 (m, 2H), 1.46-1.31 (m, 12H).

### Step 3.1: tert-butyl N-[(3R,6S)-6-(benzyloxycarbonylaminocarbamoyl)tetrahydropyran-3-yl]carbamate

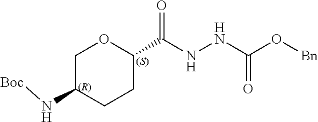

To a solution of (2S,5R)-5-(tert-butoxycarbonylamino)tetrahydropyran-2-carboxylic acid (710 mg, 2.89 mmol) and DIPEA (1.0 mL, 5.79 mmol) in dry DMF (7 mL) was added HATU (1.21 g, 3.18 mmol). The solution was stirred for 10 minutes. Benzyl N-aminocarbamate (529 mg, 3.18 mmol) was then added by portions and the reaction mixture was stirred at r.t. for 1 h.

The reaction was quenched with water (20 mL) and stirred vigorously for 10 min. The mixture was filtered to collect the off-white precipitate, which was further dried in a high vacuum oven to afford tert-butyl N-[(3R,6S)-6-(benzyloxycarbonylaminocarbamoyl)tetrahydropyran-3-yl]carbamate (950 mg, 2.20 mmol, 76% Yield) as an off-white powder. ¹NMR (400 MHz, DMSO-d₆) δ 9.60 (s, 1H), 9.12 (s, 1H), 7.35 (d, J=15.1 Hz, 5H), 6.82 (d, J=7.1 Hz, 1H), 5.07 (s, 2H), 3.88 (d, J=6.1 Hz, 1H), 3.74 (d, J=9.7 Hz, 1H), 3.08-2.95 (m, 1H), 1.99-1.78 (m, 2H), 1.57-1.29 (m, 12H). M/Z: 416 [M+Na], ESI+, RT=1.09 min (S1).

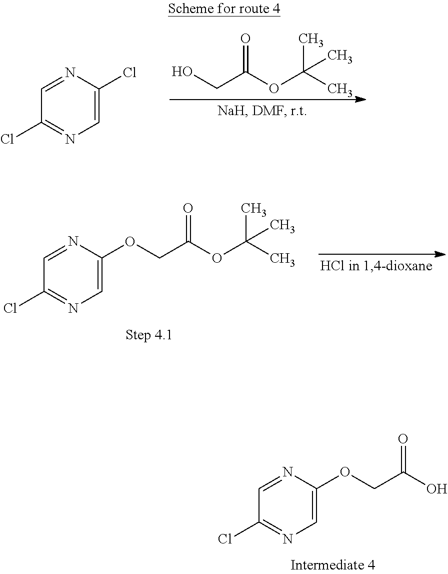

### Intermediate 4: 2-(5-chloropyrazin-2-yl)oxyacetic acid

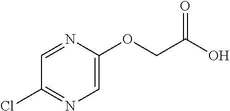 [SMILES: O=C(O)COc1cnc(Cl)cn1]

4 M hydrogen chloride (10 mL, 40.0 mmol) in 1,4-dioxane was added to tert-butyl 2-(5-chloropyrazin-2-yl)oxyacetate (269 mg, 1.09 mmol) at r.t. and stirred for 72 h. The mixture was evaporated to dryness. The residue was purified by flash chromatography using a C18-12 g KP-Ultra SNAP cartridge eluting with a solution of MeCN (+0.1% formic acid) in water (+0.1% formic acid) (10 to 100%) to afford 2-(5-chloropyrazin-2-yl)oxyacetic acid (120 mg, 0.630 mmol, 58% Yield) as a white solid. ¹H NMR (500 MHz, DMSO-d₆) δ 8.36 (d, J=1.3 Hz, 1H), 8.29 (d, J=1.3 Hz, 1H), 4.89 (s, 2H). M/Z: 187, 189 [M−H], ESI−, RT=0.76 min (S1).

### Step 4.1: tert-butyl 2-(5-chloropyrazin-2-yl)oxyacetate

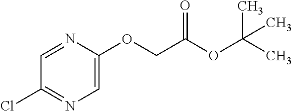 [SMILES: CC(C)(C)OC(=O)COc1cnc(Cl)cn1]

To a solution of tert-butyl 2-hydroxyacetate (0.049 mL, 3.69 mmol) in dry DMF (5 mL) at r.t. was added sodium hydride (89 mg, 3.69 mmol) by portion over 5 min. Additional DMF (5 mL) was added to the suspension and stirred for 30 min. 2,5-dichloropyrazine (500 mg, 3.36 mmol) was then added dropwise and the reaction mixture stirred at r.t. for 3 h. The reaction mixture was slowly diluted with water (50 mL) and extracted with EtOAc (2×30 mL). The combined organic layer was washed with brine (30 mL), dried over Na₂SO₄, filtered and evaporated to dryness. The residue was purified using Method 1 to afford tert-butyl 2-(5-chloropyrazin-2-yl)oxyacetate (269 mg, 1.09 mmol, 32% Yield) as a white solid. ¹H NMR (500 MHz, Chloroform-d) δ 8.12 (d, J=1.0 Hz, 1H), 8.05 (d, J=1.0 Hz, 1H), 4.78 (s, 2H), 1.47 (s, 9H). M/Z: 245, 247 [M+H], ESI+, RT=1.18 min (S1).

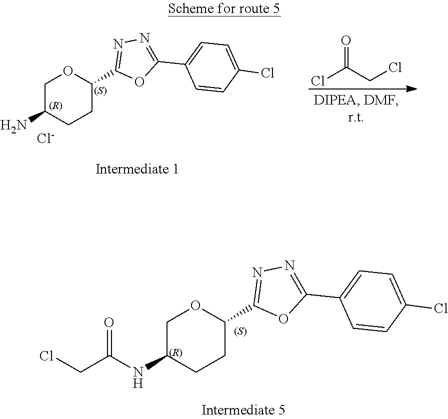

### Intermediate 5: 2-chloro-N-[(3R,6S)-6-[5-(4-chlorophenyl)-1,3,4-oxadiazol-2-yl]tetrahydropyran-3-yl]acetamide

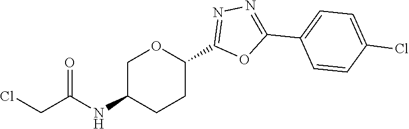 [SMILES: O=C(CCl)N[C@@H]1CC[C@@H](c2nnc(-c3ccc(Cl)cc3)o2)OC1]

A solution of [(3R,6S)-6-[5-(4-chlorophenyl)-1,3,4-oxadiazol-2-yl]tetrahydropyran-3-yl]ammonium chloride (250 mg, 0.791 mmol) and DIPEA (0.28 mL, 1.58 mmol) in DMF (3 mL) was stirred for 5 min. The reaction mixture was cooled to 0° C. before the addition of 2-chloroacetyl chloride (89 mg, 0.791 mmol) in DMF (3 mL). The reaction mixture was warmed to r.t. and stirred for 1.5 h. Water (10 mL) was added, the reaction mixture was filtered under vacuum and further rinsed with water to afford 2-chloro-N-[(3R,6S)-6-[5-(4-chlorophenyl)-1,3,4-oxadiazol-2-yl]tetrahydropyran-3-yl]acetamide (146 mg, 0.344 mmol, 44% Yield) was obtained as a brown solid. ¹H NMR (500 MHz, DMSO-d₆) δ 8.27 (d, J=7.6 Hz, 1H), 8.06-8.01 (m, 2H), 7.71-7.67 (m, 2H), 4.84 (dd, J=10.6, 2.6 Hz, 1H), 4.06 (d, J=1.3 Hz, 2H), 3.98-3.92 (m, 1H), 3.85-3.75 (m, 1H), 3.50-3.25 (m, 1H), 2.16 (dt, J=10.5, 4.3 Hz, 1H), 2.08-1.96 (m, 2H), 1.72-1.62 (m, 1H). M/Z: 356, 358 [M+H], ESI+, RT=1.03 min (S1).

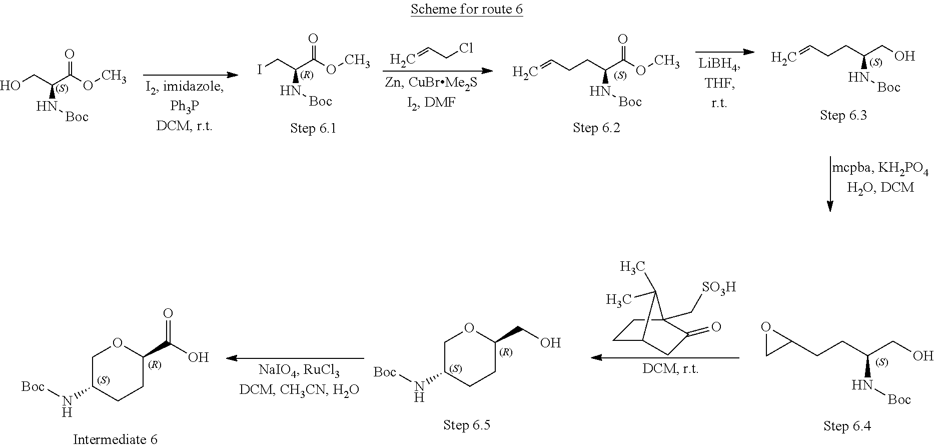

### Intermediate 6: (2R,5S)-5-(tert-butoxycarbonylamino)tetrahydropyran-2-carboxylic acid

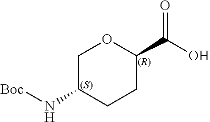

A solution of tert-butyl N-[(3S,6R)-6-(hydroxymethyl)tetrahydropyran-3-yl]carbamate (657 mg, 2.84 mmol) in DCM (5 mL), acetonitrile (5 mL) and water (7 mL) was vigorously stirred whilst cooling to 0° C. Sodium periodate (1.22 g, 5.68 mmol) and ruthenium(3+) trichloride (0.027 g, 0.13 mmol) were added and the reaction stirred at this temperature for 3 h. EtOAc (10 mL) was added and the mixture filtered. Methanol was added and the solution was filtrated. A solution of 10% sodium bisulfite (10 ml) was added and the pH was adjusted to 2 with 1 M HCl. The aqueous layer was separated, extracted with EtOAc. The organic layers were combined, dried over MgSO₄ and concentrated under reduced pressure. The residue was taken up in saturated NaHCO₃ (10 mL) and extracted with EtOAc (2×10 mL). The aqueous layer was acidified to pH 2 with 1 M HCl and extracted with EtOAc (4×10 mL), the organic layers were combined, dried over MgSO₄ and concentrated under reduced pressure. The residue was triturated with 1:2 TBME/heptane (100 mL), filtered, dried in vacuo to afford (2R,5S)-5-(tert-butoxycarbonylamino)tetrahydropyran-2-carboxylic acid (375 mg, 1.53 mmol, 54% Yield) as a yellow powder. ¹H NMR (400 MHz, Chloroform-d) δ 4.57-4.15 (m, 2H), 4.13-3.85 (m, 2H), 3.79-3.39 (m, 1H), 3.15 (t, J=10.6 Hz, 1H), 2.27-2.03 (m, 2H), 1.87-1.62 (m, 1H), 1.44 (s, 10H).

### Step 6.1: methyl (2R)-2-(tert-butoxycarbonylamino)-3-iodo-propanoate

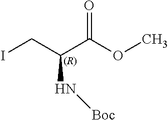

Imidazole (4.27 g, 62.8 mmol) was added to a solution of triphenylphosphane (16.46 g, 62.8 mmol) in DCM (200 mL) at r.t. and after complete dissolution cooled to 0° C. under N₂ atmosphere. Molecular iodine (15.93 g, 62.8 mmol) was added portion wise over 20 min. The solution was warmed to r.t., stirred for 10 min and cooled back to 0° C. A solution of methyl (2{S})-2-(tert-butoxycarbonylamino)-3-hydroxy-propanoate (10.59 g, 48.3 mmol) in DCM (50 mL) was added dropwise over 1 h. The reaction is stirred at 0° C. for 1 h, allowed to warm to r.t. and stirred for a further 1.5 h. The reaction mixture was filtered through a silica plug (75 g) eluting with 1:1 ether:heptanes and solvents evaporated. The residue was purified by chromatography on silica gel eluting 0-30% TBME in heptanes to give a clear oil. After crystallization from heptane, the solid was collected by filtration and dried in vacuo to afford methyl (2R)-2-(tert-butoxycarbonylamino)-3-iodo-propanoate (11.46 g, 33.1 mmol, 69% Yield). ¹H NMR (500 MHz, Chloroform-d) δ 5.34 (d, J=5.9 Hz, 1H), 4.56-4.46 (m, 1H), 3.80 (s, 3H), 3.63-3.49 (m, 2H), 1.46 (s, 9H).

### Step 6.2: methyl (2S)-2-(tert-butoxycarbonylamino)hex-5-enoate

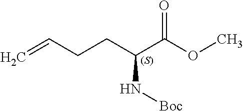

Zinc (1.96 g, 30.0 mmol) and molecular iodine (76 mg, 0.299 mmol) were added to a 3-neck flask fitted with a thermometer. The flask was evacuated and heated with a heat gun for 10 min, then flushed with N₂ and the process repeated twice. After cooling to r.t., dry DMF (1 mL) was added and the slurry was cooled to 0° C. A solution of methyl (2{R})-2-(tert-butoxycarbonylamino)-3-iodo-propanoate (3.29 g, 10.0 mmol) in DMF (6.5 mL) was added dropwise over 10 min and the reaction mixture stirred at r.t. for 1 h.

A second 3-neck flask fitted with a thermometer was charged with bromocopper methylsulfanylmethane (207 mg, 1.00 mmol) and gently heated under vacuum with a heat gun while the colour changed from off-white to pale green. After cooling to r.t., DMF (6.5 mL) and 3-chloroprop-1-ene (0.81 mL, 10.0 mmol) were added. The flask was cooled to −15° C. and the zinc reagent was added dropwise. The reaction mixture was allowed to warm to r.t. and stirred for 18 h. EtOAc (75 mL) was added and the mixture stirred for 15 min, diluted with further EtOAc (75 mL), washed with 5% Na₂S₂O₃ (2×25 mL), water (2×25 mL), brine (25 mL), dried over Na₂SO₄ and concentrated under reduced pressure. The residue was purified by chromatography on silica gel eluting 0-50% TBME in heptane to give methyl (2S)-2-(tert-butoxycarbonylamino)hex-5-enoate (1.96 g, 7.67 mmol, 77% Yield) as a clear oil. ¹H NMR (500 MHz, Chloroform-d) δ 5.79 (ddt, J=16.9, 10.2, 6.6 Hz, 1H), 5.08-4.95 (m, 3H), 4.36-4.27 (m, 1H), 3.74 (s, 3H), 2.17-2.05 (m, 2H), 1.90 (dq, J=13.5, 7.4 Hz, 1H), 1.71 (dq, J=14.4, 8.0 Hz, 1H), 1.44 (s, 9H).

### Step 6.3: tert-butyl N-[(1S)-1-(hydroxymethyl)pent-4-enyl]carbamate

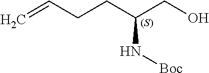

To a suspension of lithium borohydride (0.17 g, 7.67 mmol) in THE (43 mL) at r.t. under N₂ atmosphere was added a solution of methyl (2S)-2-(tert-butoxycarbonylamino)hex-5-enoate (95%, 1.96 g, 7.67 mmol) in THE (14 mL) and the resulting solution stirred at r.t. for 18 h. Water was added and the mixture extracted with EtOAc, the organic layers were combined, washed with brine, dried over Na₂SO₄ and concentrated under reduced pressure to give tert-butyl N-[(1S)-1-(hydroxymethyl)pent-4-enyl]carbamate (1.85 g, 7.73 mmol, 100% Yield) as a colourless oil. ¹H NMR (500 MHz, Chloroform-d) δ 5.86-5.76 (m, 1H), 5.07-4.95 (m, 2H), 4.63 (s, 1H), 3.66 (s, 2H), 3.56 (dd, J=10.1, 5.0 Hz, 1H), 2.20-2.06 (m, J=7.3, 6.8 Hz, 2H), 1.68-1.48 (m, 3H), 1.45 (s, 9H).

### Step 6.4: tert-butyl N-[(1S)-1-(hydroxymethyl)-3-(oxiran-2-yl)propyl]carbamate

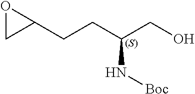

A solution of tert-butyl N-[(1S)-1-(hydroxymethyl)pent-4-enyl]carbamate (1.85 g, 7.73 mmol) in DCM (30 mL) was added to a solution of potassium phoshate (4.04 g, 23.2 mmol) in water (40 mL) and vigorously stirred at r.t. 3-chlorobenzenecarboperoxoic acid (1.78 g, 7.73 mmol) was added and stirring continued for 18 h. The layers were separated and the aqueous extracted with DCM (50 mL). The organic layers were combined, dried over Na₂SO₄ and concentrated in vacuo. The residue was purified by chromatography on silica gel eluting 0-100% EtOAc in heptane to afford tert-butyl N-[(1S)-1-(hydroxymethyl)-3-(oxiran-2-yl)propyl]carbamate (1.32 g, 4.58 mmol, 59% Yield) as a clear oil. ¹H NMR (500 MHz, Chloroform-d) δ 4.72 (d, J=31.2 Hz, 1H), 3.72-3.50 (m, 3H), 2.98-2.90 (m, 1H), 2.57-2.43 (m, 1H), 2.45-2.20 (m, 1H), 1.80-1.51 (m, 4H), 1.44 (s, 10H).

### Step 6.5: tert-butyl N-[(3S,6R)-6-(hydroxymethyl)tetrahydropyran-3-yl]carbamate

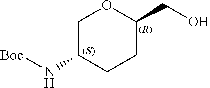

(7,7-dimethyl-2-oxobicyclo[2.2.1]hept-1-yl)methanesulfonic acid (262 mg, 1.13 mmol) was added to a solution of tert-butyl N-[(LS)-1-(hydroxymethyl)-3-(oxiran-2-yl)propyl]carbamate (3.48 g, 11.3 mmol) in DCM (75 mL) and the resulting solution stirred at r.t. for 18 h. The reaction mixture was poured into an aqueous solution of NaHCO₃ and the layers separated. The organic layer was dried over Na₂SO₄ and concentrated under reduced pressure. The residue was purified by flash chromatography on silica gel eluting 0-100% EtOAc in heptane to give an off-white powder. The solid was triturated with heptane to afford tert-butyl N-[(3S,6R)-6-(hydroxymethyl)tetrahydropyran-3-yl]carbamate (662 mg, 2.86 mmol, 25% Yield) as an off-white powder. ¹H NMR (500 MHz, Chloroform-d) δ 4.26 (s, 1H), 4.11 (ddd, J=10.7, 4.7, 2.1 Hz, 1H), 3.60 (ddd, J=11.2, 7.9, 3.1 Hz, 2H), 3.51 (ddd, J=11.5, 7.1, 4.5 Hz, 1H), 3.36 (dtd, J=10.3, 5.5, 2.7 Hz, 1H), 3.02 (t, J=10.7 Hz, 1H), 2.16-1.96 (m, 2H), 1.51-1.36 (m, 10H), 1.29 (qd, J=12.5, 4.2 Hz, 1H)

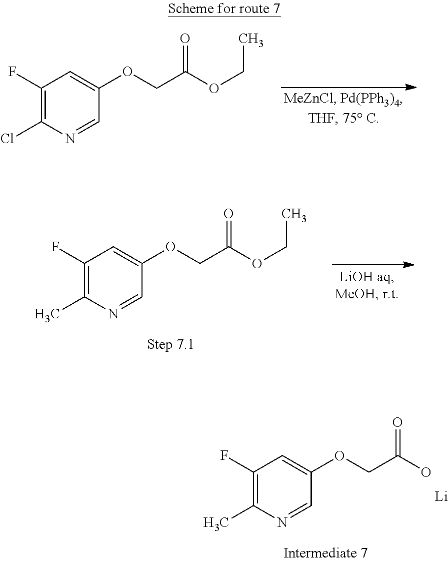

### Intermediate 7: lithium 2-[(5-fluoro-6-methyl-3-pyridyl)oxy]acetate

To a solution of ethyl 2-[(5-fluoro-6-methyl-3-pyridyl)oxy]acetate (0.50 g, 2.35 mmol) in methanol (5 mL) at r.t. was added 2 M hydroxylithium (2.3 mL, 4.69 mmol) and stirred at r.t. overnight before evaporating to dryness. The solid was suspended in acetonitrile (10 mL), and evaporated to dryness to give lithium 2-[(5-fluoro-6-methyl-3-pyridyl)oxy]acetate (630 mg, 2.34 mmol, 100% Yield) as a white solid. ¹H NMR (400 MHz, DMSO-d₆) δ 8.01-7.84 (m, 1H), 7.08-7.00 (m, 1H), 4.20-4.11 (m, 2H), 3.20-3.13 (m, 1H), 2.35-2.29 (m, 3H). M/Z: 186 [M+H]+, RT=0.4-0.6 min (S4).

### Step 7.1: ethyl 2-[(5-fluoro-6-methyl-3-pyridyl)oxy]acetate

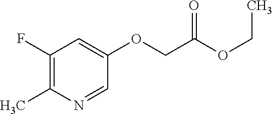

To a degassed solution of ethyl 2-[(6-chloro-5-fluoro-3-pyridyl)oxy]acetate (97%, 2.60 g, 10.8 mmol) in anhydrous THF (30 mL) at r.t. under a nitrogen atmosphere was added palladium triphenylphosphane (0.80 g, 0.692 mmol) and stirred. 2 M chloro(methyl)zinc (6.5 mL, 13.0 mmol) in THF was then added and stirred for 5 min. The reaction mixture was heated to 75° C., stirred overnight and allowed to cool to RT. The reaction mixture was quenched with ammonium chloride solution (20 mL), diluted with water (100 mL), and extracted with EtOAc (2×50 mL). The organics were dried over sodium sulfate, filtered and evaporated to dryness. Purification by flash chromatography (Biotage Isolera, C18 120 g KP-Ultra SNAP cartridge) eluting with a solution of MeCN (+0.1% formic acid) in water (+0.1% formic acid) (10 to 100%) followed by evaporation gave ethyl 2-[(5-fluoro-6-methyl-3-pyridyl)oxy]acetate (1.69 g, 7.69 mmol, 71% Yield) as an off-white solid. ¹H NMR (400 MHz, Chloroform-d) δ 8.05 (d, J=2.4 Hz, 1H), 6.94 (dd, J=10.4, 2.5 Hz, 1H), 4.63 (s, 2H), 4.27 (q, J=7.1 Hz, 2H), 2.45 (d, J=2.9 Hz, 3H), 1.30 (t, J=7.1 Hz, 3H). M/Z: 214 [M+H]+, RT=0.98 (S1).

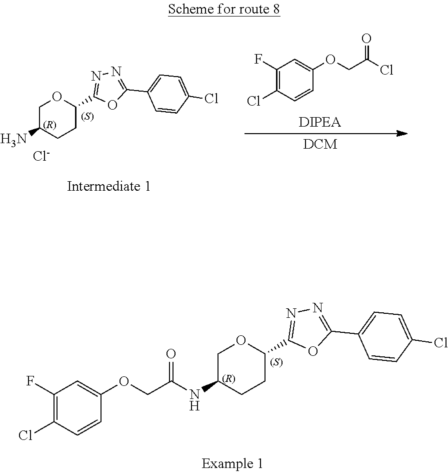

### Example 1: 2-(4-chloro-3-fluoro-phenoxy)-N-[(3R,6S)-6-[5-(4-chlorophenyl)-1,3,4-oxadiazol-2-yl]tetrahydropyran-3-yl]acetamide

To a solution of [(3R,6S)-6-[5-(4-chlorophenyl)-1,3,4-oxadiazol-2-yl]tetrahydropyran-3-yl]ammonium chloride (78 mg, 0.241 mmol) in DCM (1.5 mL) was added DIPEA (0.17 mL, 0.964 mmol) followed by a solution of 2-(4-chloro-3-fluoro-phenoxy)acetyl chloride (0.11 g, 0.482 mmol) in DCM (1 mL) dropwise at r.t.. After stirring for 5 min, the reaction mixture was diluted with 1 M aqueous hydrogen chloride solution and DCM. The organic layer was isolated and washed sequentially with 1 M NaOH solution and brine, dried (MgSO₄), filtered and concentrated in vacuo. The residual material was purified by column chromatography (silica gel, eluting with heptanes-EtOAc, 1:0 to 0:1) to afford 2-(4-chloro-3-fluoro-phenoxy)-N-[(3R,6S)-6-[5-(4-chlorophenyl)-1,3,4-oxadiazol-2-yl]tetrahydropyran-3-yl]acetamide (106 mg, 0.22 mmol, 92% Yield) as an off-white solid. ¹H NMR (500 MHz, DMSO-d₆) δ 8.12 (d, J=7.8 Hz, 1H), 8.00-8.06 (m, 2H), 7.67-7.73 (m, 2H), 7.51 (t, J=8.9 Hz, 1H), 7.09 (dd, J=11.4, 2.8 Hz, 1H), 6.83-6.91 (m, 1H), 4.82 (dd, J=10.7, 2.6 Hz, 1H), 4.56 (s, 2H), 3.84-4.00 (m, 2H), 3.38 (t, J=10.2 Hz, 1H), 2.12-2.22 (m, 1H), 1.95-2.08 (m, 2H), 1.68-1.80 (m, 1H). M/Z: 466[M+H], ESI+, RT=4.20 min (S1).

Compounds in Table 1 were synthesized according to the general route 8 as exemplified by Example 1 using the corresponding intermediates.

TABLE 1

| | | | | LCMS | |
|---|---|---|---|---|---|
| Ex | Structure | Name | Intermediates | data | 1H NMR |
| 1 | 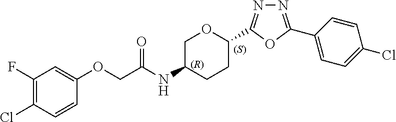 | 2-(4-chloro-3- fluorophenoxy)- N-[(3R,6S)- 6-[5-(4- chlorophenyl)- 1,3,4- oxadiazol-2- yl]oxan-3- | [(3R,6S)-6-[5- (4- chlorophenyl)- 1,3,4-oxadiazol- 2-yl] tetrahydropyran- 3-yl]ammonium chloride | M/Z: 465.95 [M + H]+, RT = 4.2 (S3) | (500 MHz, DMSO-d₆) δ 8.12 (d, J = 7.8, 1H), 8.00- 8.06 (m, 2H), 7.67- 7.73 (m, 2H), 7.51 (t, J = 8.9, 1H), 7.09 (dd, J = 11.4, 2.8,1H), 6.83- 6.91 (m, 1H), 4.82 (dd, J = 10.7, 2.6, 1H), |
| | | yl]acetamide | (Intermediate 1) | | 3.84-4.00 (m, 2H), 3.38 |
| | | | | | (t, J = 10.2, 1H), 2.12- |
| | | | | | 2.22 (m, 1H), 1.95-2.08 |
| | | | | | (m, 2H), 1.68-1.80 (m, |
| | | | | | 1H) |
| 2 | 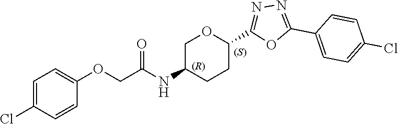 | 2-(4- chlorophenoxy)- N-[(3R,6S)- 6-[5-(4- chlorophenyl)- 1,3,4- oxadiazol-2- yl]oxan-3- | [(3R,6S)-6-[5- (4- chlorophenyl)- 1,3,4-oxadiazol- 2-yl] tetrahydropyran- 3-yl]ammonium chloride | M/Z: 448, 450 [M + H]+, RT = 3.62 (S2) | (500 MHz, DMSO-d6) 8.09 (d, J = 7.8 Hz, 1H), 8.06-7.99 (m, 2H), 7.71- 7.65 (m, 2H), 7.37-7.31 (m, 2H), 7.03-6.95 (m, 2H), 4.80 (dd, J = 10.7, 2.6 Hz, 1H), 4.55-4.45 (m, 2H), 3.97-3.83 (m, |
| | | yl]acetamide | (Intermediate 1) | | 2H), 3.37 (t, J = 10.2 Hz, |
| | | | | | 1H), 2.20-2.12 (m, 1H), |
| | | | | | 2.06-1.95 (m, 2H), 1.80- |
| | | | | | 1.67 (m, 1H). |
| 3 | 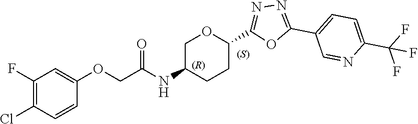 | 2-(4-chloro-3- fluorophenoxy)- N-[(3R,6S)- 6-{5-[6- (trifluoromethyl) pyridin-3-yl]- 1,3,4- oxadiazol-2- | (3R,6S)-6-{5-[6- (trifluoromethyl) pyridin-3-yl]- 1,3,4-oxadiazol- 2-yl}oxan-3- amine hydrochloride from 6- | M/Z: 501 [M + H]+, RT = 3.49 (S2) | (500 MHz, Chloroform- d) 9.43 (s, 1H), 8.59 (dd, J = 8.1, 1.7 Hz, 1H), 7.89 (d, J = 8.2 Hz, 1H), 7.38 (dd, J = 8.6 Hz, 1H), 6.81 (dd, J = 10.2, 2.8 Hz, 1H), 6.75-6.70 (m, 1H), 6.44 (d, J = 8.0 Hz, 1H), |
| | | yl}oxan-3- | (trifluoromethyl) | | 4.86 (dd, J = 9.1, 3.7 Hz, |
| | | yl]acetamide | pyridine-3- | | 1H), 4.51 (s, 2H), 4.29- |
| | | | carbohydrazide | | 4.21 (m, 2H), 3.49-3.42 |
| | | | [CAS 386715- | | (m, 1H), 2.39-2.20 (m, |
| | | | 32-8] following | | 3H), 1.81-1.71 (m, 1H). |
| | | | Route 1 | | |
| 4 | 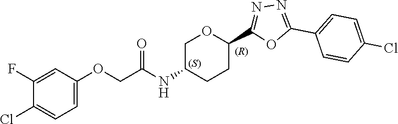 | 2-(4-chloro-3- fluorophenoxy)- N-[(3S,6R)- 6-[5-(4- chlorophenyl)- 1,3,4- oxadiazol-2- yl]oxan-3- | (3S,6R)-6-[5-(4- chlorophenyl)- 1,3,4-oxadiazol- 2-yl]oxan-3- amine hydrochloride from (2R,5S)-5- (tert- | M/Z: 466, 468, RT = 3.71 (S2) | (400 MHz, DMSO-d6) δ 8.11 (d, J = 7.7 Hz, 1H), 8.06-8.00 (m, 2H), 7.72- 7.67 (m,2H), 7.51 (t, J = 8.9 Hz, 1H), 7.08 (dd, J = 11.4, 2.8 Hz, 1H), 6.88- 6.82 (m, 1H), 4.81 (dd, J = 10.7, 2.6 Hz, 1H), |
| | | yl]acetamide | butoxy carbonyl | | 4.55 (s, 2H), 3.99-3.82 |
| | | | amino) | | (m, 2H), 3.37 (t, J = 10.1 |
| | | | tetrahydropyran- | | Hz, 1H), 2.16 (dd, J = |
| | | | 2- | | 9.9, 3.9 Hz, 1H), 2.08- |
| | | | carboxylic acid | | 1.94 (m, 2H), 1.81-1.66 |
| | | | [Intermediate 6] | | (m, 1H). |
| | | | following | | |
| | | | Route 1 | | |
| 5 | 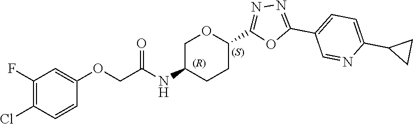 | 2-(4-chloro-3- fluorophenoxy)- N-[(3R,6S)- 6-[5-(6- cyclopropylpy- ridin-3-yl)- 1,3,4- oxadiazol-2- | (3R,6S)-6-[5-(6- cyclopropylpyri- din-3-yl)-1,3,4- oxadiazol-2- yl]oxan-3-amine hydrochloride from tert-butyl N-[(3R,6S)-6- | M/Z: 473 [M + H]+, RT = 3.38 (S2) | (500 MHz,CDCl3) 9.08 (d, J = 1.7 Hz, 1H), 8.17 (dd, J = 8.2, 2.3 Hz, 1H), 7.35 (dd, J = 8.6 Hz, 1H), 7.29-7.26 (m, 1H), 6.78 (dd, J = 10.2, 2.9 Hz, 1H), 6.70 (ddd, J = 8.9, 2.8, 1.2 Hz, 1H), 6.43 (d, |
| | | yl]oxan-3- | (hydrazine- | | J = 7.8 Hz, 1H), 4.83- |
| | | yl]acetamide | carbonyl)tetrahy- | | 4.79 (m, 1H), 4.48 (s, |
| | | | dropyran-3- | | 2H), 4.25-4.16 (m,2H), |
| | | | yl]carbamate | | 3.46-3.39 (m, 1H), 2.34- |
| | | | (Intermediate 3) | | 2.27 (m, 1H), 2.25- |
| | | | and 6- | | 2.19 (m, 2H), 2.14-2.08 |
| | | | cyclopropyl- | | (m, 1H), 1.76-1.67 (m, |
| | | | pyridine-3- | | 1H), 1.17-1.07 (m, 4H). |
| | | | carboxylic acid | | |
| | | | [CAS 75893-75- | | |
| | | | 3] following | | |
| | | | Route 1 | | |
| 6 | 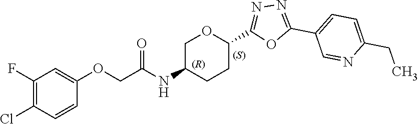 | 2-(4-chloro-3- fluorophenoxy)- N-[(3R,6S)- 6-[5-(6- ethylpyridin- 3-yl)-1,3,4- oxadiazol-2- yl]oxan-3- | (3R,6S)-6-[5-(6- ethylpyridin-3- yl)-1,3,4- oxadiazol-2- yl]oxan-3- aminium chloride from 6- ethylpyridine-3- | M/Z: 461 [M + H]+, RT = 3.13 (S2) | (500 MHz,CDCl3) 9.19 (d, J = 2.1 Hz, 1H), 8.27 (dd, J = 8.2, 2.3 Hz, 1H), 7.38-7.29 (m, 2H), 6.78 (dd, J = 10.2, 2.8 Hz, 1H), 6.70 (ddd, J = 8.9, 2.8, 1.0 Hz, 1H), 6.43 (d, J = 7.8 Hz, 1H), 4.84- |
| | | yl]acetamide | carboxylic | | 4.79 (m, 1H), 4.48 (s, |
| | | | acid[CAS | | 2H), 4.26-4.16 (m, 2H), |
| | | | 802828-81-5] | | 3.47- 3.38 (m, 1H), 2.92 |
| | | | and tert-butyl | | (q, J = 7.6 Hz, 2H), 2.35- |
| | | | N-[(3R,6S)-6- | | 2.28 (m, 1H), 2.26- |
| | | | (hydrazine- | | 2.20 (m, 2H), 1.77-1.68 |
| | | | carbonyl) | | (m, 1H), 1.35 (t, J = 7.6 |
| | | | tetrahydro- | | Hz, 3H). |
| | | | pyran-3- | | |
| | | | yl]carbamate | | |
| | | | (intermediate 3) | | |
| | | | following | | |
| | | | Route 1 | | |

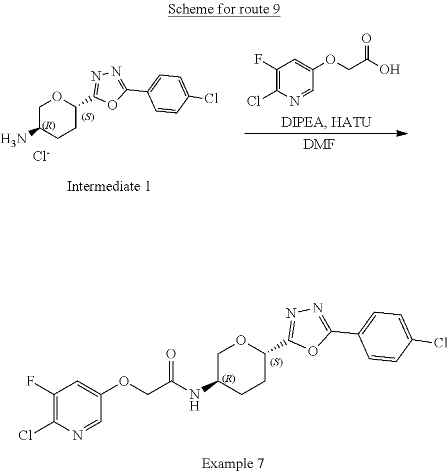

### Example 7: 2-[(6-chloro-5-fluoro-3-pyridyl)oxy]-N-[3R,6S)-6-[5-(4-chlorophenyl)-1,3,4-oxadiazol-2-yl]tetrahydropyran-3-yl]acetamide

To a solution of 2-[(6-chloro-5-fluoro-3-pyridyl)oxy]acetic acid (36 mg, 0.174 mmol), HATU (66 mg, 0.174 mmol) and N-ethyl-N-isopropyl-propan-2-amine (0.055 mL, 0.316 mmol) in dry DMF (2 mL) was added [(3R,6S)-6-[5-(4-chlorophenyl)-1,3,4-oxadiazol-2-yl]tetrahydropyran-3-yl]ammonium chloride (50 mg, 0.158 mmol). The mixture was stirred at r.t. for 60 min. The reaction mixture was then diluted with EtOAc, washed with water, followed by saturated aqueous solution of NaHCO₃ (20 mL), dried over sodium sulfate, filtered and evaporated to dryness. The solid was then purified by preparative HPLC (Method 1) to afford 2-[(6-chloro-5-fluoro-3-pyridyl)oxy]-N-[(3R,6S)-6-[5-(4-chlorophenyl)-1,3,4-oxadiazol-2-yl]tetrahydropyran-3-yl]acetamide (37 mg, 0.0784 mmol, 50% Yield) as a white powder. ¹HNMR(500 MHz, DMSO-d₆) δ=8.17 (d, J=7.8, 1H), 8.08 (d, J=2.6, 1H), 8.06-8.01 (m, 2H), 7.71 (dd, J=10.3, 2.6, 1H), 7.70-7.66 (m, 2H), 4.87-4.76 (m, 1H), 4.67 (d, J=1.9, 2H), 3.97-3.91 (m, 1H), 3.92-3.84 (m, 1H), 3.40-3.37 (m, 1H), 2.16 (d, J=13.7, 1H), 2.10-1.95 (m, 2H), 1.77-1.67 (m, 1H). M/Z: 467, 469 [M+H], ESI+, RT=3.35 min (S2).

Compounds in Table 2 were synthesized according to the general route 9 as exemplified by Example 7 using the corresponding intermediates.

TABLE 2

| | | | | LCMS | |
|---|---|---|---|---|---|
| Ex | Structure | Name | Intermediates | data | 1HNMR |
| 7 | 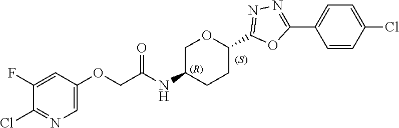 | 2-[(6-chloro-5- fluoropyridin-3- yl)oxy]-N- [(3R,6S)-6-[5-(4- chlorophenyl)- 1,3,4-oxadiazol- 2-yl]oxan-3- yl]acetamide | 2-[(6-chloro- 5-fluoro-3- pyridyl)oxy] acetic acid (Intermediate 2) | M/Z: 467, 469 [M + H]+, RT = 3.35 (S2) | (500 MHz, DMSO-d6) 8.17 (d, J = 7.8, 1H), 8.08 (d, J = 2.6, 1H), 8.06- 8.01 (m, 2H), 7.71 (dd, J = 10.3, 2.6, 1H), 7.70-7.66 (m, 2H), 4.87- 4.76 (m, 1H), 4.67 (d, J = 1.9, 2H), 3.97- |
| | | | | | 3.91 (m, 1H), 3.92- |
| | | | | | 3.84 (m, 1H), 3.40-3.37 |
| | | | | | (m, 1H), 2.16 (d, J = 13.7, |
| | | | | | 1H), 2.10-1.95 (m, 2H), |
| | | | | | 1.77-1.67 (m, 1H). |
| 8 | 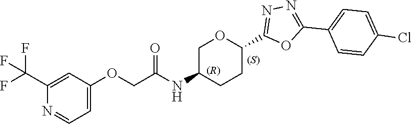 | N-[(3R,6S)-6- [5-(4- chlorophenyl)- 1,3,4-oxadiazol- 2-yl]oxan-3-yl]- 2-{[2- (trifluoromethyl) pyridin-4- | 2-[[2- (trifluorometh yl)-4- pyridyl]oxy] acetic acid from 2- (trifluoro- methyl)pyridin- | M/Z: 483, 485 [M + H]+, RT = 3.22 (S2) | (500 MHz, DMSO-d6) 8.60 (d, J = 5.7, 1H), 8.22 (d, J = 7.7, 1H), 8.05- 8.00 (m, 2H), 7.75-7.64 (m, 2H), 7.45 (d, J = 2.4, 1H), 7.26 (dd, J = 5.7, 2.5, 1H), 4.83 (dd, J = 10.6, 2.6, 1H), 4.76 (d, J = 3.0, |
| | | yl]oxy}acetamide | 4-ol[CAS | | 2H), |
| | | | 1876148-59-2] | | 4.00-3.84 (m, 2H), 3.37 |
| | | | following | | (s, 1H), 2.21-2.12 (m, |
| | | | route 2 | | 1H), 2.09-1.97 (m, 2H), |
| | | | | | 1.80-1.68 (m, 1H). |
| 9 | 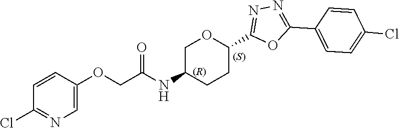 | N-[(3R,6S)-6- [5-(4- chlorophenyl)- 1,3,4-oxadiazol- 2-yl]oxan-3-yl]- 2-[(6- chloropyridin-3- yl)oxy] acetamide | 2-[(6-chloro- 3- pyridyl)oxy] acetic acid from 6- chloropyridin- 3-ol [CAS 105-36-2] following | M/Z: 449, 451 [M + H]+, RT = 3.08 (S2) | (500 MHz, DMSO-d6) 8.20-8.12 (m, 2H), 8.04 (d, J = 8.6 Hz, 2H), 7.69 (d, J = 8.6 Hz, 2H), 7.49 (dd, J = 8.8, 2.9 Hz, 1H), 7.46 (d, J = 8.6 Hz, 1H), 4.82 (dd, J = 10.7, 2.5 Hz, 1H), 4.66-4.59 (m, 2H), 3.94 (dd, J = 10.6, |
| | | | route 2 | | 3.3 Hz, 1H), 3.92-3.84 |
| | | | | | (m, 1H), 3.40-3.34 (m, |
| | | | | | 1H), 2.21-2.13 (m, 1H), |
| | | | | | 2.07-1.96 (m, 2H), 1.79- |
| | | | | | 1.68 (m, 1H). |
| 10 | 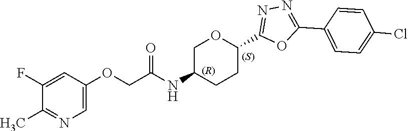 | N-[(3R,6S)-6- [5-(4- chlorophenyl)- 1,3,4-oxadiazol- 2-yl]oxan-3-yl]- 2-[(5-fluoro-6- methylpyridin- 3-yl)oxy] acetamide | lithium 2-[(5- fluoro-6- methyl-3- pyridyl)oxy] acetate (Intermediate 7) | M/Z: 447, 449 [M + H]+, RT = 2.96 (S2) | (400 MHz, DMSO-d6) 8.18-8.12(m, 1H), 8.11- 8.07 (m, 1H), 8.06-7.99 (m, 2H), 7.72-7.65 (m, 2H), 7.40-7.31 (m, 1H), 4.85-4.77 (m, 1H), 4.60 (s, 2H), 3.98-3.81 (m, 2H), 3.42-3.37 (m, 1H), 2.39-2.34 (m, 3H), 2.20- |
| | | | | | 2.12 (m, 1H), 2.08- |
| | | | | | 1.93 (m, 2H), 1.80-1.66 |
| | | | | | (m, 1H). |
| 11 | 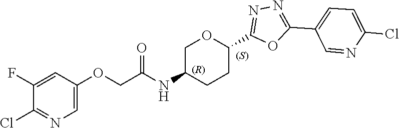 | 2-[(6-chloro-5- fluoropyridin-3- yl)oxy]-N- [(3R,6S)-6-[5-(6- chloropyridin-3- yl)-1,3,4- oxadiazol-2- yl]oxan-3- yl]acetamide | (3R,6S)-6-[5- (6- chloropyridin- 3-yl)-1,3,4- oxadiazol-2- yl]oxan-3- amine hydrochloride using 6- | M/Z: 468, 470 [M + H]+, RT = 2.84 (S2) | (500 MHz, DMSO-d6) 9.03 (d, J = 2.4 Hz, 1H), 8.44 (dd, J = 8.4, 2.4 Hz, 1H), 8.19 (d, J = 7.8 Hz, 1H), 8.09 (d, J = 2.6 Hz, 1H), 7.78 (d, J = 8.4 Hz, 1H), 7.72 (dd, J = 10.3, 2.6 Hz, 1H), 4.85 (dd, J = 10.6, 2.5 Hz, 1H), 4.68 |
| | | | methylpyridine- | | (s, 2H), 3.96 (dd, J = |
| | | | 3-carbohy- | | 10.6, 3.4 Hz, 1H), 3.93- |
| | | | drazide [CAS | | 3.84 (m, 1H), 3.40 (s, |
| | | | 197079-25-7] | | 1H),2.18(dd, J = 10.1, |
| | | | following | | 3.8 Hz, 1H), 2.09-1.97 |
| | | | Route 1 and 2- | | (m, 2H), 1.80-1.68 (m, |
| | | | [(6-chloro-5- | | 1H). |
| | | | fluoro-3- | | |
| | | | pyridyl)oxy] | | |
| | | | acetic acid | | |
| | | | (intermediate | | |
| | | | 2) | | |
| 12 | 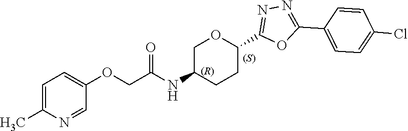 | N-[(3R,6S)-6- [5-(4- chlorophenyl)- 1,3,4-oxadiazol- 2-yl]oxan-3-yl]- 2-[(6- methylpyridin- 3-yl)oxy] acetamide | lithium;2-[(6- methyl-3- pyridyl)oxy] acetate from 6- methylpyridin- 3-ol following route 2 | M/Z: 429 [M + H]+, RT = 1.89 (S2) | (500 MHz, DMSO-d6) δ 8.49 (d, J = 2.9 Hz, 1H), 8.21 (d, J = 7.8 Hz, 1H), 8.05-8.00 (m, 2H), 7.98 (dd, J = 8.9, 2.6 Hz, 1H), 7.74 (d, J = 8.9 Hz, 1H), 7.71-7.66 (m, 2H), 4.83 (dd, J = 10.6, 2.5 Hz, 1H), 4.75 (d, J = 2.6 Hz, |
| | | | | | 2H), 3.95 (dd, J = 10.1, |
| | | | | | 3.8 Hz, 1H), 3.87 (ddt, |
| | | | | | J = 15.8, 12.1, 5.8 Hz, |
| | | | | | 2H), 2.60 (s, 3H), 2.20- |
| | | | | | 2.14 (m, 1H), 2.06-1.97 |
| | | | | | (m, 2H), 1.72 (qd, J = |
| | | | | | 13.6, 12.9, 4.3 Hz, 1H) |
| 13 | 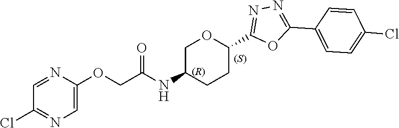 | N-[(3R,6S)-6- [5-(4- chlorophenyl)- 1,3,4-oxadiazol- 2-yl]oxan-3-yl]- 2-[(5- chloropyrazin- 2- | 2-(5- chloropyrazin- 2-yl)oxyacetic acid (Intermediate 4) | M/Z: 450, 452,454 [M + H]+. RT = 3.14 (S2) | (500 MHz, DMSO-d6) 8.37-8.33 (m, 1H), 8.28 (s, 1H), 8.17-8.09 (m, 1H), 8.05-8.01 (m, 2H), 7.72- 7.67 (m, 2H), 4.84- 4.76 (m, 3H), 3.96-3.89 (m, 1H), 3.88-3.78 (m, 1H), 3.50-3.29 (m, 1H), |
| | | yl)oxy] | | | 2.19-2.12 (m, 1H), 2.08- |
| | | acctamide | | | 1.93 (m, 2H), 1.73- |
| | | | | | 1.62 (m, 1H). |
| 14 | 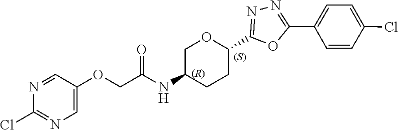 | N-[(3R,6S)-6- [5-(4- chlorophenyl)- 1,3,4-oxadiazol- 2-yl]oxan-3-yl]- 2-[(2- chloropyrimidin- 5-yl)oxy] acetamide | (2-(2- chloropyri- midin-5- yl)oxyacetic acid from 2- chloropy- rimidin-5-ol following route 2 | M/Z: 450, 452 [M + H]+, RT = 2.9 (S2) | (500 MHz, DMSO-d6) 8.54 (s, 2H), 8.20 (d, J = 7.8, 1H), 8.08- 7.97 (m, 2H), 7.74-7.61 (m, 2H), 4.82 (dd, J = 10.7, 2.5, 1H), 4.74 (d, J = 2.3, 2H), 3.95 (dd, J = 10.1, 3.8, 1H), 3.90-3.82 (m, 1H), 3.37 (d, J = 10.4, |
| | | | | | 1H), 2.21-2.12 (m, 1H), |
| | | | | | 2.08-1.96 (m, 2H), 1.78- |
| | | | | | 1.64 (m, 1H). |
| 15 | 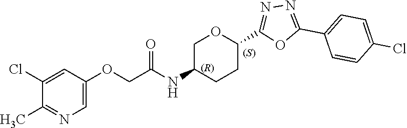 | 2-[(5-chloro-6- methylpyridin- 3-yl)oxy]-N- [(3R,6S)-6-[5- (4- chlorophenyl)- 1,3,4-oxadiazol- 2-yl]oxan-3- yl]acetamide | 2-[(5-chloro- 6-methyl-3- pyridyl)oxy] acetic acid from 5-chloro- 6-methyl- pyridin-3-ol [CAS 51984- 63-5] | M/Z: 463, 465 [M + H]+, RT = 3.25 (S2) | (500 MHz, DMSO-d6) 8.19 (d, J = 2.6, 1H), 8.14 (d, J = 7.6, 1H), 8.08- 7.97 (m, 2H), 7.80-7.63 (m, 2H), 7.54 (d, J = 2.6, 1H), 4.85-4.77 (m, 1H), 4.61 (d, J = 1.5, 2H), 3.93 (d, J = 10.6, 3H), 2.47 (s, 3H), 2.15 (s, 1H), 2.02 |
| | | | following | | (s, 2H), 1.75 (s, 1H). |
| | | | route 2 | | |
| 16 | 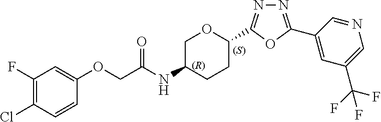 | 2-(4-chloro-3- fluorophenoxy)- N-[(3R,6S)-6- {5-[5- (trifluoromethyl) pyridin-3-yl]- 1,3,4-oxadiazol- 2-yl}oxan-3- yl]acetamide | From (3R,6S)- 6-{5-[5- (trifluoro- methyl)pyridin- 3-yl]-1,3,4- oxadiazol-2- yl}oxan-3- amine hydrochloride | M/Z: 501, 503 [M + H]+, RT = 3.42 (S2) | (500 MHz, DMSO-d6) δ = 9.47 (d, J = 1.9, 1H), 9.27-9.20 (m, 1H), 8.69 (s, 1H), 8.12 (d, J = 7.8, 1H), 7.50 (t, J = 8.9, 1H), 7.08 (dd, J = 11.4, 2.8, 1H), 6.94-6.81 (m, 1H), 4.86 (dd, J = 10.7, 2.6, 1H), 4.55 (d, J = 1.1, 2H), |
| | | | from 5- | | 3.99-3.84 (m, 2H), 3.41- |
| | | | Trifluoro- | | 3.39 (m, 1H), 2.27- |
| | | | methylnicotinic | | 2.11 (m, 1H), 2.10-1.98 |
| | | | acid [CAS | | (m,2H), 1.82-1.68 (m, |
| | | | 131747-40-5] | | 1H). |
| | | | following | | |
| | | | Route 1 | | |
| 17 | 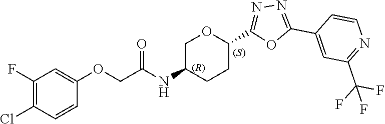 | 2-(4-chloro-3- fluorophcnoxy)- N-[(3R,6S)-6- {5-[2- (trifluoromethyl )pyridin-4-yl]- 1,3,4-oxadiazol- 2-yl}oxan-3- yl]acetamide | (3R,6S)-6-{5- [2- (trifluorometh yl)pyridin-4- yll-1,3,4- oxadiazol-2- yl}oxan-3- amine hydrochloride | M/Z: 501, 503 [M + H]+, RT = 3.52 (S2) | (400 MHz, DMSO-d6) δ = 9.04 (d, J = 5.0, 1H), 8.34 (s, 1H), 8.31 (d, J = 5.0, 1H), 8.13 (d, J = 7.8, 1H), 7.51 (t, J = 8.9, 1H), 7.09 (dd, J = 11.4, 2.8, 1H), 6.87 (m, J = 9.0, 2.8, 1.1, 1H), 4.88 (dd, J = 10.7, |
| | | | using 2- | | 2.6, 1H), 4.56 (s, 2H), |
| | | | (trifluorometh | | 4.00-3.86 (m, 2H), 3.41- |
| | | | yl)isonicotinic | | 3.39 (m, 1H), 2.25- |
| | | | acid [CAS | | 2.15 (m, 1H), 2.10-2.01 |
| | | | 131747-41-6] | | (m, 2H), 1.83-1.69 (m, |
| | | | and | | 1H). |
| | | | Intermediate 3 | | |
| | | | following | | |
| | | | route 1 | | |

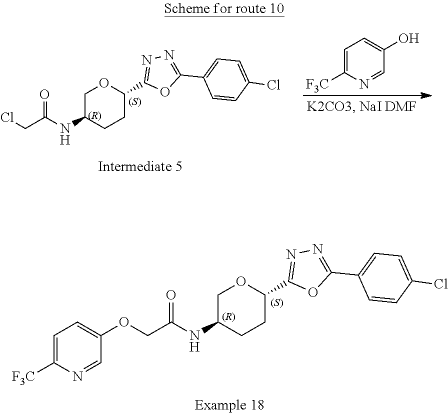

### Example 18: N-[13R,6S)-6-[5-(4-chlorophenyl)-1,3,4-oxadiazol-2-yl]tetrahydropyran-3-yl]-2-[[6-(trifluoromethyl)-3-pyridyl]oxy]acetamide

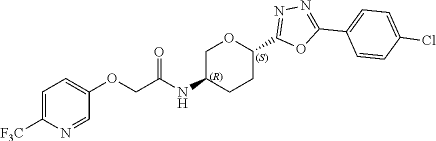

A solution of 2-chloro-N-[(3R,6S)-6-[5-(4-chiorophenyl)-1,3,4-oxadiazol-2-yl]tetrahydropyran-3-yl]acetamide (84%, 70 mg, 0.165 mmol), dipotassium carbonate (46 mg, 0.330 mmol), sodium iodide (37 mg, 0.248 mmol) and 6-(trifluoromethyl)pyridin-3-ol (27 mg, 0.165 mmol) in dry DMF (1 mL) under N₂ was stirred at 40° C. for 4 h. Water was added and the precipitate formed was filtered under vacuum. The residue was purified by column chromatography on silica gel column using EtOAc/Heptane (40-100%) as eluent to afford N-[(3R,6S)-6-[5-(4-chlorophenyl)-1,3,4-oxadiazol-2-yl]tetrahydropyran-3-yl]-2-[[6-(trifluoromethyl)-3-pyridyl]oxy]acetamide (41 mg, 0.082 mmol, 50% Yield) as a white solid. ¹H NMR (500 MHz, DMSO-d6) δ 8.48 (d, J=2.8 Hz, 1H), 8.23 (d, J=7.8 Hz, 1H), 8.06-8.01 (m, 2H), 7.88 (d, J=8.7 Hz, 1H), 7.71-7.66 (m, 2H), 7.58 (dd, J=8.7, 2.8 Hz, 1H), 4.83 (dd, J=10.7, 2.5 Hz, 1H), 4.74 (d, J=1.8 Hz, 2H), 3.98-3.93 (m, 1H), 3.93-3.85 (m, 1H), 3.40 (s, 1H), 2.17 (dt, J=10.2, 2.5 Hz, 1H), 2.08-1.97 (m, 2H), 1.78-1.69 (m, 1H). M/Z. 483, 485 [M+H]+, RT=3.37 (S2).

Compounds in Table 3 were synthesized according to the general route 10 as exemplified by Example 18 using the corresponding intermediates.

TABLE 3

| | | | | LCMS | |
|---|---|---|---|---|---|
| Ex | Structure | Name | Intermediates | data | 1HNMR |
| 18 | 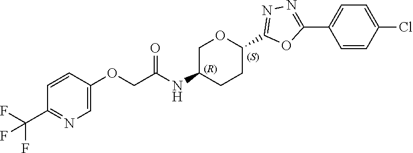 | N-[3R,6S)-6- [5-(4- chlorophenyl)- 1,3,4- oxadiazol-2- yl] tetrahydropyran- 3-yl]-2-[[6- trifluoromethyl)- 3-pyridyl]oxy] | 6- (trifluoromethyl) pyridin-3-ol | M/Z: 483, 485 [M + H]+, RT = 3.37 (S2) | (500 MHz, DMSO-d6) δ 8.48 (d, J = 2.8 Hz, 1H), 8.23 (d, J = 7.8 Hz, 1H), 8.06-8.01 (m, 2H), 7.88 (d, J = 8.7 Hz, 1H), 7.71- 7.66 (m, 2H), 7.58 (dd, J = 8.7, 2.8 Hz, 1H), 4.83 (dd, J = 10.7, 2.5 Hz, 1H), 4.74 (d, J = |
| | | acetamide | | | 1.8 Hz, 2H), 3.98- |
| | | | | | 3.93 (m, 1H), 3.93- |
| | | | | | 3.85 (m, 1H), 3.40 (s, |
| | | | | | 1H), 2.17 (dt, J = 10.2, |
| | | | | | 2.5 Hz, 1H), 2.08- |
| | | | | | 1.97 (m, 2H), 1.78- |
| | | | | | 1.69 (m, 1H). |
| 19 | 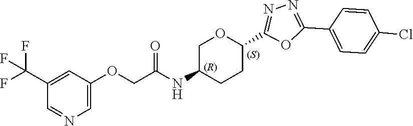 | N-[(3R,6S)-6- [5-(4- chlorophenyl)- 1,3,4- oxadiazol-2- yl]oxan-3-yl]- 2-{[5- (trifluoromethyl) pyridin-3- | 5- (trifluoromethyl) pyridin-3-ol | M/Z: 483, 485 [M + H]+, RT = 3.31 (S2) | (500 MHz, DMSO-d6) δ 8.64 (d, J = 2.7 Hz, 1H), 8.59 (d, J = 2.4 Hz, 1H), 8.21 (d, J = 7.8 Hz, 1H), 8.06- 8.01 (m, 2H), 7.76 (t, J = 2.1 Hz, 1H), 7.71- 7.67 (m, 2H), 4.83 (dd, J = 10.7, 2.6 Hz, 1H), |
| | | yl]oxy} | | | 4.75 (d, J = 2.3 Hz, |
| | | acetamide | | | 2H), 3.97-3.92 (m, |
| | | | | | 1H), 3.92-3.85 (m, |
| | | | | | 1H), 3.40 (s, 1H), |
| | | | | | 2.21-2.14(m, 1H), |
| | | | | | 2.07-1.97 (m, 2H), |
| | | | | | 1.79-1.68 (m, 1H). |

### II Biological Assay

HEK-ATF4 High Content Imaging Assay

Example compounds were tested in the HEK-ATF4 High Content Imaging assay to assess their pharmacological potency to prevent Tunicamycin induced ISR. Wild-type HEK293 cells were plated in 384-well imaging assay plates at a density of 12,000 cells per well in growth medium (containing DMEM/F12, 10% FBS, 2 mM L-Glutamine, 100 U/mL Penicillin—100 μg/mL Streptomycin) and incubated at 37° C., 5% CO₂. 24-hrs later, the medium was changed to 50 μl assay medium per well (DMEM/F12, 0.3% FBS, 2 mM L-Glutamine, 100 U/mL Penicillin—100 μg/mL Streptomycin). Example compounds were serially diluted in dimethyl sulfoxide (DMSO), spotted into intermediate plates and prediluted with assay medium containing 3.3 μM Tunicamycin to give an 11-fold excess of final assay concentration. In addition to the example compound testing area, the plates also contained multiples of control wells for assay normalization purposes, wells, containing Tunicamycin but no example compounds (High control), as well as wells containing neither example compound nor Tunicamycin (Low control). The assay was started by transferring 5 μl from the intermediate plate into the assay plates, followed by incubation for 6 hrs at 37° C., 5% CO₂. Subsequently, cells were fixed (4% PFA in PBS, 20 min at room temperature) and submitted to indirect ATF4 immunofluorescence staining (primary antibody rabbit anti ATF4, clone D4B8, Cell Signaling Technologies; secondary antibody Alexa Fluor 488 goat anti-rabbit IgG (H+L), Thermofisher Scientific). Nuclei were stained using Hoechst dye (Thermofisher Scientific), and plates were imaged on an Opera Phenix High Content imaging platform equipped with 405 nm and 488 nm excitation. Finally, images were analyzed using script based algorithms. The main readout HEK-ATF4 monitored the ATF4 signal ratio between nucleus and cytoplasm. Tunicamycin induced an increase in the overall ATF4 ratio signal, which was prevented by ISR modulating example compounds. In addition, HEK-CellCount readout was derived from counting the number of stained nuclei corresponding to healthy cells. This readout served as an internal toxicity control. The example compounds herein did not produce significant reduction in CellCount.

Activity of the tested example compounds is provided in Table T5 as follows:

+++=IC50 1-500 nM; ++=IC50>500-2000 nM; +=IC50>2000-15000 nM.

TABLE T5

| Example | |
|---|---|
| number | Activity |
| 1 | +++ |
| 2 | +++ |
| 3 | +++ |
| 4 | +++ |
| 5 | +++ |
| 6 | ++ |
| 7 | +++ |
| 8 | ++ |
| 9 | +++ |
| 10 | ++ |
| 11 | + |
| 12 | + |
| 13 | ++ |
| 14 | ++ |
| 15 | +++ |
| 16 | ++ |
| 17 | +++ |
| 18 | +++ |
| 19 | ++ |

### REFERENCES

(1) Pakos-Zebrucka K, Koryga I, Mnich K, Ljujic M, Samali A, Gorman A M. The integrated stress response. EMBO Rep. 2016 October; 17(10):1374-1395. Epub 2016 Sep. 14.
(2) Wek R C, Jiang H Y, Anthony T G. Coping with stress: eIF2 kinases and translational control. Biochem Soc Trans. 2006 February; 34 (Pt 1):7-11.
(3) Donnelly N, Gorman A M, Gupta S, Samali A. The eIF2alpha kinases: their structures and functions. Cell Mol Life Sci. 201 30ct; 70(19):3493-511
(4) Jackson R J, Hellen C U, Pestova T V. The mechanism of eukaryotic translation initiation and principles of its regulation. Nat Rev Mol Cell Biol. 2010 February; 11(2):113-27
(5) Lomakin I B, Steitz T A. The initiation of mammalian protein synthesis and mRNA scanning mechanism. Nature. 2013 Aug. 15; 500(7462):307-11
(6) Pain V M. Initiation of protein synthesis in eukaryotic cells. Eur J Biochem. 1996 Mar. 15; 236(3):747-71
(7) Pavitt G D. Regulation of translation initiation factor eIF2B at the hub of the integrated stress response. Wiley Interdiscip Rev RNA. 2018 November; 9(6): e1491.
(8) Krishnamoorthy T, Pavitt G D, Zhang F, Dever T E, Hinnebusch A G. Tight binding of the phosphorylated alpha subunit of initiation factor 2 (eIF2alpha) to the regulatory subunits of guanine nucleotide exchange factor eIF2B is required for inhibition of translation initiation. Mol Cell Biol. 2001 August; 21(15):5018-30.
(9) Hinnebusch, A. G., Ivanov, I. P., & Sonenberg, N. (2016). Translational control by 5′-untranslated regions of eukaryotic mRNAs. Science, 352(6292), 1413-1416.
(10) Young, S. K., & Wek, R. C. (2016). Upstream open reading frames differentially regulate gene-specific translation in the integrated stress response. The Journal of Biological Chemistry, 291(33), 16927-16935.
(11) Lin J H, Li H, Zhang Y, Ron D, Walter P (2009) Divergent effects of PERK and IRE1 signaling on cell viability. PLoS ONE 4: e4170
(12) Tabas I, Ron D. Nat Cell Biol. 2011 March; 13(3):184-90. Integrating the mechanisms of apoptosis induced by endoplasmic reticulum stress.
(13) Shore G C, Papa F R, Oakes S A. Curr Opin Cell Biol. 2011 April; 23(2):143-9. Signaling cell death from the endoplasmic reticulum stress response.
(14) Bi M, Naczki C, Koritzinsky M, Fels D, Blais J, Hu N, Harding H, Novoa I, Varia M, Raleigh J, Scheuner D, Kaufman R J, Bell J, Ron D, Wouters B G, Koumenis C. EMBO J. 2005 Oct. 5; 24(19):3470-81 ER stress-regulated translation increases tolerance to extreme hypoxia and promotes tumor growth.
(15) Bobrovnikova-Marjon E, Grigoriadou C, Pytel D, Zhang F, Ye J, Koumenis C, Cavener D, Diehl J A. Oncogene. 2010 Jul. 8; 29(27):3881-95 PERK promotes cancer cell proliferation and tumor growth by limiting oxidative DNA damage.
(16) Avivar-Valderas A, Salas E, Bobrovnikova-Marjon E, Diehl J A, Nagi C, Debnath J, Aguirre-Ghiso J A. Mol Cell Biol. 2011 September; 31(17):3616-29. PERK integrates autophagy and oxidative stress responses to promote survival during extracellular matrix detachment.
(17) Blais, J. D.; Addison, C. L.; Edge, R.; Falls, T.; Zhao, H.; Kishore, W.; Koumenis, C.; Harding, H. P.; Ron, D.; Holcik, M.; Bell, J. C. Mol. Cell. Biol. 2006, 26, 9517 -9532.PERK-dependent translational regulation promotes tumor cell adaptation and angiogenesis in response to hypoxic stress.
(18) Taalab Y M, Ibrahim N, Maher A, Hassan M, Mohamed W, Moustafa A A, Salama M, Johar D, Bernstein L. Rev Neurosci. 2018 Jun. 27; 29(4):387-415. Mechanisms of disordered neurodegenerative function: concepts and facts about the different roles of the protein kinase RNA-like endoplasmic reticulum kinase (PERK).
(19) Remondelli P, Renna M. Front Mol Neurosci. 2017 Jun. 16; 10:187. The Endoplasmic Reticulum Unfolded Protein Response in Neurodegenerative Disorders and Its Potential Therapeutic Significance.
(20) Halliday M, Mallucci G R. Neuropathol Appl Neurobiol. 2015 June; 41(4):414-27.Review: Modulating the unfolded protein response to prevent neurodegeneration and enhance memory.
(21) Halliday M, Radford H, Sekine Y, Moreno J, Verity N, le Quesne J, Ortori C A, Barrett D A, Fromont C, Fischer P M, Harding H P, Ron D, Mallucci G R. Cell Death Dis. 2015 Mar. 5; 6: e1672.Partial restoration of protein synthesis rates by the small molecule ISRIB prevents neurodegeneration without pancreatic toxicity.
(22) Moreno J A, Radford H, Peretti D, Steinert J R, Verity N, Martin M G, Halliday M, Morgan J, Dinsdale D, Ortori C A, Barrett D A, Tsaytler P, Bertolotti A, Willis A E, Bushell M, Mallucci G R. Nature 2012; 485: 507-11. Sustained translational repression by eIF2alpha-P mediates prion neurodegeneration.
(23) Skopkova M, Hennig F, Shin B S, Turner C E, Stanikova D, Brennerova K, Stanik J, Fischer U, Henden L, Müller U, Steinberger D, Leshinsky-Silver E, Bottani A, Kurdiova T, Ukropec J, Nyitrayova 0, Kolnikova M, Klimes I, Borck G, Bahlo M, Haas S A, Kim J R, Lotspeich-Cole L E, Gasperikova D, Dever T E, Kalscheuer V M. Hum Mutat. 2017 April; 38(4):409-425. EIF2S3 Mutations Associated with Severe X-Linked Intellectual Disability Syndrome MEHMO.
(24) Hamilton E M C, van der Lei H D W, Vermeulen G, Gerver J A M, Lourengo C M, Naidu S, Mierzewska H, Gemke RJBJ, de Vet H C W, Uitdehaag B M J, Lissenberg-Witte B I; VWM Research Group, van der Knaap M S. Ann Neurol. 2018 August; 84(2):274-288. Natural History of Vanishing White Matter.
(25) Bugiani M, Vuong C, Breur M, van der Knaap M S. Brain Pathol. 2018 May; 28(3):408-421. Vanishing white matter: a leukodystrophy due to astrocytic dysfunction.
(26) Wong Y L, LeBon L, Edalji R, Lim H B, Sun C, Sidrauski C. Elife. 2018 Feb. 28; 7. The small molecule ISRIB rescues the stability and activity of Vanishing White Matter Disease eIF2B mutant complexes.
(27) Wong Y L, LeBon L, Basso A M, Kohlhaas K L, Nikkel A L, Robb H M, Donnelly-Roberts D L, Prakash J, Swensen A M, Rubinstein N D, Krishnan S, McAllister F E, Haste N V, O'Brien J J, Roy M, Ireland A, Frost J M, Shi L, Riedmaier S, Martin K, Dart M J, Sidrauski C. Elife. 2019 Jan. 9; 8. eIF2B activator prevents neurological defects caused by a chronic integrated stress response.
(28) Nguyen H G, Conn C S, Kye Y, Xue L, Forester C M, Cowan J E, Hsieh A C, Cunningham J T, Truillet C, Tameire F, Evans M J, Evans C P, Yang J C, Hann B, Koumenis C, Walter P, Carroll P R, Ruggero D. Sci Transl Med. 2018 May 2; 10 (439). Development of a stress response therapy targeting aggressive prostate cancer.

1-21. (canceled)

22. A compound of formula (I) 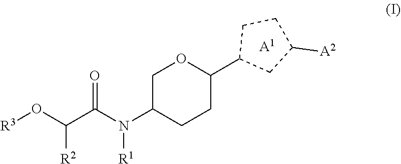
or a pharmaceutically acceptable salt, solvate, hydrate, tautomer, or stereoisomer thereof, wherein
A¹ is C₅ cycloalkylene, C₅ cycloalkenylene, or a nitrogen ring atom containing 5-membered heterocyclene, wherein A¹ is optionally substituted with one or more R⁴, which are the same or different;
each R⁴ is independently halogen, CN, OR⁵, or oxo (═O) where the ring is at least partially saturated or C₁₋₆ alkyl, wherein C₁₋₆ alkyl is optionally substituted with one or more halogen, which are the same or different;
R⁵ is H or C₁₋₆ alkyl, wherein C₁₋₆ alkyl is optionally substituted with one or more halogen, which are the same or different;
A² is phenyl or 5- to 6-membered aromatic heterocyclyl, wherein A² is optionally substituted with one or more R⁶, which are the same or different;
each R⁶ is independently OH, O(C₁₋₆ alkyl), halogen, CN, cyclopropyl, C₁₋₆alkyl, C₂₋₆ alkenyl, or C₂₋₆ alkynyl, wherein cyclopropyl, C₁₋₆ alkyl, C₂₋₆ alkenyl, and C₂₋₆ alkynyl are optionally substituted with one or more halogen, which are the same or different; or
two R⁶ are joined to form together with atoms to which they are attached a ring A^{2a};
A^{2a} is phenyl, C₃₋₇ cycloalkyl, or 3- to 7-membered heterocyclyl, wherein A^{2a} is optionally substituted with one or more R⁷, which are the same or different;
each R⁷ is independently C₁₋₆ alkyl, C₂₋₆ alkenyl, or C₂₋₆ alkynyl, wherein C₁₋₆ alkyl, C₂₋₆ alkenyl, and C₂₋₆ alkynyl are optionally substituted with one or more halogen, which are the same or different;
R¹ is H or C₁₋₄alkyl, wherein C₁₋₄ alkyl is optionally substituted with one or more halogen, which are the same or different;
R² is H or C₁₋₄ alkyl, wherein C₁₋₄ alkyl is optionally substituted with one or more halogen, which are the same or different; and
R³ is A³; or
R² and R³ are joined to form a 3,4-dihydro-2H-1-benzopyran ring, which is optionally substituted with one or more R⁸, which are the same or different;
A³ is phenyl or 5- to 6-membered aromatic heterocyclyl, wherein A³ is optionally substituted with one or more R⁸, which are the same or different;
each R⁸ is independently halogen, CN, C(O)OR⁹, OR⁹, C(O)R⁹, C(O)N(R⁹R^{9a}), S(O)₂N(R⁹R^{9a}), S(O)N(R⁹R^{9a}), S(O)₂R⁹, S(O)R⁹, N(R⁹)S(O)₂N(R^{9a}R^{9b}), SR⁹, N(R⁹R^{9a}), NO₂, OC(O)R⁹, N(R⁹)C(O)R^{9a}, N(R⁹)S(O)₂R^{9a}, N(R⁹)S(O)R^{9a}, N(R⁹)C(O)OR^{9a}, N(R⁹)C(O)N(R^{9a}R^{9b}), OC(O)N(R⁹R^{9a}), oxo (═O) where the ring is at least partially saturated, C₁₋₆ alkyl, C₂₋₆ alkenyl, or C₂₋₆ alkynyl, wherein C₁₋₆ alkyl, C₂₋₆ alkenyl, and C₂₋₆ alkynyl are optionally substituted with one or more R¹⁰, which are the same or different;
R⁹, R^{9a}, and R^{9b} are independently selected from the group consisting of H, C₁₋₆ alkyl, C₂₋₆ alkenyl, and C₂₋₆ alkynyl, wherein C₁₋₆ alkyl, C₂₋₆ alkenyl, and C₂₋₆ alkynyl are optionally substituted with one or more halogen, which are the same or different;
each R¹⁰ is independently halogen, CN, C(O)OR¹¹, OR¹¹, C(O)R¹¹, C(O)N(R¹¹R^{11a}), S(O)₂N(R¹¹R^{11a}), S(O)N(R¹¹R^{11a}), S(O)₂R¹¹, S(O)R¹¹, N(R¹¹)S(O)₂N(R^{11a}R^{11b}), SR¹¹, N(R¹¹R^{11a}), NO₂, OC(O)R¹¹, N(R¹¹)C(O)R¹¹, N(R¹¹)SO₂R^{11a}, N(R¹¹)S(O)R^{11a}, N(R¹¹)C(O)N(R^{11a}R^{11b}), N(R¹¹)C(O)OR^{11a}, or OC(O)N(R¹¹R^{11a});
R¹¹, R^{11a}, and R^{11b} are independently selected from the group consisting of H, C₁₋₆ alkyl, C₂₋₆ alkenyl, and C_{2- 6} alkynyl, wherein C₁₋₆ alkyl, C₂₋₆ alkenyl, and C₂₋₆ alkynyl are optionally substituted with one or more halogen, which are the same or different.

23. The compound of claim 22 or a pharmaceutically acceptable salt, solvate, hydrate, tautomer, or stereoisomer thereof, wherein A¹ is a nitrogen ring atom containing 5-membered heterocyclene and wherein A¹ is optionally substituted with one or more R⁴, which are the same or different.

24. The compound of claim 22 or a pharmaceutically acceptable salt, solvate, hydrate, tautomer, or stereoisomer thereof, wherein A¹ is a nitrogen ring atom containing 5-membered heterocyclene selected from the group of bivalent heterocycles consisting of oxadiazole, imidazole, imidazolidine, pyrazole and triazole, and wherein A¹ is optionally substituted with one or more R⁴, which are the same or different.

25. The compound of claim 22 or a pharmaceutically acceptable salt, solvate, hydrate, tautomer, or stereoisomer thereof, wherein A¹ is unsubstituted or substituted with one or two R⁴, which are the same or different.

26. The compound of claim 22 or a pharmaceutically acceptable salt, solvate, hydrate, tautomer, or stereoisomer thereof, wherein R⁴ is oxo, where the ring is at least partly saturated.

27. The compound of claim 22 or a pharmaceutically acceptable salt, solvate, hydrate, tautomer, or stereoisomer thereof, wherein A¹ is

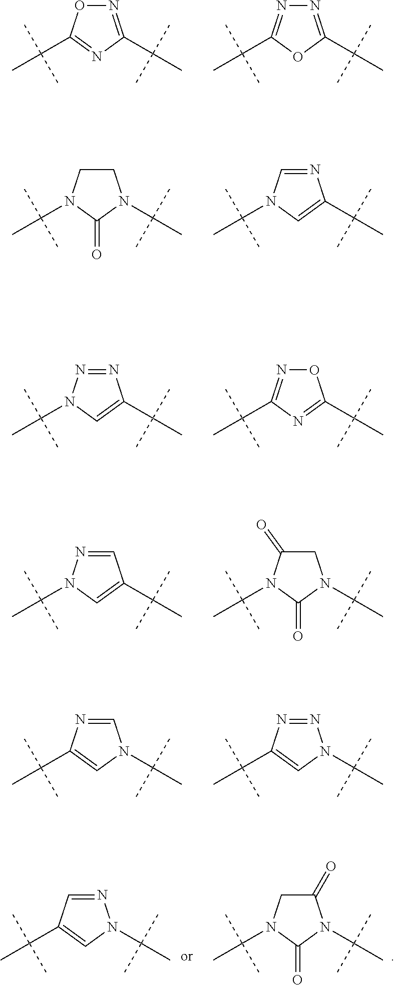

28. The compound of claim 22 or a pharmaceutically acceptable salt, solvate, hydrate, tautomer, or stereoisomer thereof, wherein A² is phenyl, pyridyl, pyrazinyl, pyridazinyl, pyrazolyl, or 1,2,4-oxadiazolyl, and wherein A² is optionally substituted with one or more R⁶, which are the same or different.

29. The compound of claim 22 or a pharmaceutically acceptable salt, solvate, hydrate, tautomer, or stereoisomer thereof, wherein A² is phenyl, pyridyl, pyrazinyl, or pyridazinyl, and wherein A² is optionally substituted with one or more R⁶, which are the same or different.

30. The compound of claim 22 or a pharmaceutically acceptable salt, solvate, hydrate, tautomer, or stereoisomer thereof, wherein A² is substituted with one or two R⁶, which are the same or different.

31. The compound of claim 22 or a pharmaceutically acceptable salt, solvate, hydrate, tautomer, or stereoisomer thereof, wherein each R⁶ is independently F, Cl, CF₃, OCH₃, CH₃, CH₂CH₃, or cyclopropyl.

32. The compound of claim 22 or a pharmaceutically acceptable salt, solvate, hydrate, tautomer, or stereoisomer thereof, wherein R² is H.

33. The compound of claim 22 or a pharmaceutically acceptable salt, solvate, hydrate, tautomer, or stereoisomer thereof, wherein R³ is A³.

34. The compound of claim 22 or a pharmaceutically acceptable salt, solvate, hydrate, tautomer, or stereoisomer thereof, wherein A³ is phenyl, pyridyl, pyrazinyl, or pyrimidazyl, and wherein A³ is optionally substituted with one or more R⁸, which are the same or different.

35. The compound of claim 22 or a pharmaceutically acceptable salt, solvate, hydrate, tautomer, or stereoisomer thereof, wherein A³ is substituted with one or two R⁸, which are the same or different.

36. The compound of claim 22 or a pharmaceutically acceptable salt, solvate, hydrate, tautomer, or stereoisomer thereof, wherein R² and R³ are joined to form the dihydrobenzopyran ring, wherein the ring is optionally substituted with one or more R⁸, which are the same or different.

37. The compound of claim 22 or a pharmaceutically acceptable salt, solvate, hydrate, tautomer, or stereoisomer thereof, wherein R⁸ is independently F, Cl, CF₃, CH═O, CH₂OH, or CH₃.

38. The compound of claim 22 or a pharmaceutically acceptable salt, solvate, hydrate, tautomer, or stereoisomer thereof, wherein the compound is 2-(4-chloro-3-fluorophenoxy)-N-[(3R,6S)-6-[5-(4-chlorophenyl)-1,3,4-oxadiazol-2-yl]oxan-3-yl]acetamide;
2-(4-chlorophenoxy)-N-[(3R,6S)-6-[5-(4-chlorophenyl)-1,3,4-oxadiazol-2-yl]oxan-3-yl]acetamide;
2-(4-chloro-3-fluorophenoxy)-N-[(3R,6S)-6-{5-[6-(trifluoromethyl)pyridin-3-yl]-1,3,4-oxadiazol-2-yl}oxan-3-yl]acetamide;
2-(4-chloro-3-fluorophenoxy)-N-[(3S,6R)-6-[5-(4-chlorophenyl)-1,3,4-oxadiazol-2-yl]oxan-3-yl]acetamide;
2-(4-chloro-3-fluorophenoxy)-N-[(3R,6S)-6-[5-(6-cyclopropylpyridin-3-yl)-1,3,4-oxadiazol-2-yl]oxan-3-yl]acetamide;
2-(4-chloro-3-fluorophenoxy)-N-[(3R,6S)-6-[5-(6-ethylpyridin-3-yl)-1,3,4-oxadiazol-2-yl]oxan-3-yl]acetamide;
2-[(6-chloro-5-fluoropyridin-3-yl)oxy]-N-[(3R,6S)-6-[5-(4-chlorophenyl)-1,3,4-oxadiazol-2-yl]oxan-3-yl]acetamide;
N-[(3R,6S)-6-[5-(4-chlorophenyl)-1,3,4-oxadiazol-2-yl]oxan-3-yl]-2-{[2-(trifluoromethyl)pyridin-4-yl]oxy}acetamide;
N-[(3R,6S)-6-[5-(4-chlorophenyl)-1,3,4-oxadiazol-2-yl]oxan-3-yl]-2-[(6-chloropyridin-3-yl)oxy]acetamide;
N-[(3R,6S)-6-[5-(4-chlorophenyl)-1,3,4-oxadiazol-2-yl]oxan-3-yl]-2-[(5-fluoro-6-methylpyridin-3-yl)oxy]acetamide;
2-[(6-chloro-5-fluoropyridin-3-yl)oxy]-N-[(3R,6S)-6-[5-(6-chloropyridin-3-yl)-1,3,4-oxadiazol-2-yl]oxan-3-yl]acetamide;
N-[(3R,6S)-6-[5-(4-chlorophenyl)-1,3,4-oxadiazol-2-yl]oxan-3-yl]-2-[(6-methylpyridin-3-yl)oxy]acetamide;
N-[(3R,6S)-6-[5-(4-chlorophenyl)-1,3,4-oxadiazol-2-yl]oxan-3-yl]-2-[(5-chloropyrazin-2-yl)oxy]acetamide;
N-[(3R,6S)-6-[5-(4-chlorophenyl)-1,3,4-oxadiazol-2-yl]oxan-3-yl]-2-[(2-chloropyrimidin-5-yl)oxy]acetamide;
2-[(5-chloro-6-methylpyridin-3-yl)oxy]-N-[(3R,6S)-6-[5-(4-chlorophenyl)-1,3,4-oxadiazol-2-yl]oxan-3-yl]acetamide;
2-(4-chloro-3-fluorophenoxy)-N-[(3R,6S)-6-{5-[5-(trifluoromethyl)pyridin-3-yl]-1,3,4-oxadiazol-2-yl}oxan-3-yl]acetamide;
2-(4-chloro-3-fluorophenoxy)-N-[(3R,6S)-6-{5-[2-(trifluoromethyl)pyridin-4-yl]-1,3,4-oxadiazol-2-yl}oxan-3-yl]acetamide;
N-[3R,6S)-6-[5-(4-chlorophenyl)-1,3,4-oxadiazol-2-yl]tetrahydropyran-3-yl]-2-[[6-(trifluoromethyl)-3-pyridyl]oxy]acetamide; or
N-[(3R,6S)-6-[5-(4-chlorophenyl)-1,3,4-oxadiazol-2-yl]oxan-3-yl]-2-{[5-(trifluoromethyl)pyridin-3-yl]oxy}acetamide.

39. A pharmaceutical composition comprising at least one compound of claim 22 or a pharmaceutically acceptable salt, solvate, hydrate, tautomer, or stereoisomer thereof, together with a pharmaceutically acceptable carrier, optionally in combination with one or more other bioactive compounds or pharmaceutical compositions.

40. A method for treating, controlling, delaying, or preventing in a mammalian patient in need of the treatment of one or more diseases or disorders associated with integrated stress response, wherein the method comprises administering to the patient a therapeutically effective amount of a compound of claim 22 or a pharmaceutically acceptable salt thereof.

41. A method for treating, controlling, delaying, or preventing in a mammalian patient in need of the treatment of one or more diseases or disorders selected from the group consisting of leukodystrophies, intellectual disability syndrome, neurodegenerative diseases and disorders, neoplastic diseases, infectious diseases, inflammatory diseases, musculoskeletal diseases, metabolic diseases, and ocular diseases, as well as diseases selected from the group consisting of organ fibrosis, chronic and acute diseases of the liver, chronic and acute diseases of the lung, chronic and acute diseases of the kidney, myocardial infarction, cardiovascular disease, arrhythmias, atherosclerosis, spinal cord injury, ischemic stroke, and neuropathic pain, wherein the method comprises administering to the patient a therapeutically effective amount of a compound of claim 22 or a pharmaceutically acceptable salt thereof.
